(19) United States
(12) Patent Application Publication
(10) Pub. No.: US 2013/0225543 A1
(43) Pub. Date: Aug. 29, 2013

(54) SPECIFIC REGULATION OF CYTOKINE LEVELS BY HDAC6 INHIBITORS

(71) Applicants: Simon S. Jones, Harvard, MA (US); Matthew Blair Jarpe, Quincy, MA (US)

(72) Inventors: Simon S. Jones, Harvard, MA (US); Matthew Blair Jarpe, Quincy, MA (US)

(73) Assignee: Acetylon Pharmaceuticals, Boston, MA (US)

(21) Appl. No.: 13/759,302

(22) Filed: Feb. 5, 2013

Related U.S. Application Data
(63) Continuation of application No. PCT/US2011/044147, filed on Jul. 15, 2011.
(60) Provisional application No. 61/371,047, filed on Aug. 5, 2010.

(51) Int. Cl. A61K 31/573; G01N 33/68; A61K 31/444; A61K 45/06; A61K 31/185; A61K 31/505

(52) U.S. Cl. CPC A61K 31/573; A61K 31/185; A61K 31/505; A61K 31/444; A61K 45/06; G01N 33/6863
USPC 514/171; 514/575; 514/275; 514/180; 514/332; 435/375; 435/6.13; 506/9

(57) ABSTRACT

The invention provides for methods of treating a disease using HDAC6 inhibitors that modulate the extracellular level of a subset of cytokines. The invention also provides for compositions for treating a disease associated with a modulation in the extracellular level of a subset of cytokines.

### PRIORITY BENEFIT

This application is a continuation of International application PCT/US2011/044147, filed on Jul. 15, 2011, which claims the benefit of U.S. Provisional application 61/371,047, filed on Aug. 5, 2010, the contents of which are incorporated herein in its entirety.

### BACKGROUND OF THE INVENTION

Post-translational modification of proteins through acetylation and deacetylation of lysine residues plays a critical role in regulating their cellular functions. At this time, eleven human HDACs have been identified (Taunton et al. Science 1996, 272, 408-411; Yang et al. J. Biol. Chem. 1997, 272, 28001-28007; Grozinger et al. Proc. Natl. Acad. Sd. U.S.A. 1999, 96, 4868-4873; Kao et al. Genes Dev. 2000, 14, 55-66. Hu et al J. Biol. Chem. 2000, 275, 15254-15264; Zhou et al. Proc. Natl. Acad. Sci U.S.A. 2001, 98, 10572-10577; Venter et al. Science 2001, 291, 1304-1351). These members are classified into four families Class I (HDAC1, 2 and 3), Class IIa (HDAC4, 5, 7 and 9), Class IIb (HDAC6 and 10) and Class IV (HDAC11). An additional seven, structurally distinct HDACs, the sirtuins, have been identified. Sirtuins use NAD as a cofactor (Lavu et al. Nature Rev. Drug Discov. 2008, 7, 841-853). Histone deacetylases are zinc-binding hydrolases, which catalyze the deacetylation of lysine residues (Haberland et al Nature Rev. Genet. 2009, 10, 32-42). Class I HDACs (HDACs 1, 2 and 3) modulate gene expression through deacetylation of the N-acetyl-lysine residues of histone proteins and other transcriptional regulators in the nucleus of the cell (Hassig et al. Curr. Opin. Chem. Biol. 1997, 1, 300-308). Class IIa HDACs can shuttle between the nucleus and the cytoplasm of the cell. However the precise mechanism of transcriptional repression by Class IIa HDACs has not been fully elucidated. HDACs participate in numerous cellular pathways that control cell shape, differentiation and proliferation, and HDAC inhibitors has been shown to be effective in treating cancer (Minucci et al. Nature Rev. Cancer 2006, 6, 38-51 and Bolden et al, Nature Rev. Drug. Discov. 2006, 5, 769-784). HDAC inhibition results in hyperacetylation of chromatin, alterations in transcription, growth arrest, and apoptosis in cancer cell lines. Early phase clinical trials with available nonselective HDAC inhibitors demonstrate responses to these compounds in hematologic malignancies including multiple myeloma, although there is significant toxicity. It is known that HDACs can regulate the acetylation levels of a wide variety of proteins in addition to histones, thereby indicating a broad role for HDACs in numerous and critical cellular pathways in addition to transcriptional regulation (Choudhary et al. Science, 2009, 325, 834-840).

To date, no small molecules are known that selectively target any particular class or individual members of the HDAC family (for example ortholog-selective HDAC inhibitors have been reported: (a) Meinke et al. J. Med. Chem. 2000, 14, 4919-4922; (b) Meinke, et al. Curr. Med. Chem. 2001, 8, 211-235). However recent studies have demonstrated that the non-selective hydroxamic acid-based inhibitors such as SAHA and ITF2357 are selective for Class I HDACs and HDAC6 but not Class IIa (Bradner et al. Nat. Chem. Biol. 2010, 6, 238-243).

HDAC6 is the main cytoplasmic deacetylase in mammalian cells and is required for aggresome formation associated with ubiquitinated protein stress. HDAC6 is involved with aggresome formation through regulation of acetylation of α-tubulin a component of microtubules and is essential for cellular viability in this context (Kawaguchi et al, Cell, 2003, 115, 727-738 and Lee et al, EMBO, 2010, 29, 969-980). HDAC6 is believed to bind ubiquitinated proteins through a zinc finger domain and is known to interact with the dynein motor complex through another discrete binding motif, which allows transport of protein complexes along the microtubules. HDAC6 possesses two catalytic deacetylase domains. It is not presently known whether the amino-terminal histone deacetylase and/or the carboxy-terminal tubulin deacetylase (TDAC) domain mediates aggresome formation. In addition HDAC6 has been shown to regulate the acetylation state of the key heat shock protein Hsp90 (Bali et al. JBC, 2005, 280, 26729-26734) and cortactin a protein involved in cell motility (Zhang et al, Mol. Cell. 2007, 27, 197-213).

Aberrant histone deacetylase activity has been linked to various neurological and neurodegenerative disorders, including stroke, Huntington's disease, Amyotrophic Lateral Sclerosis and Alzheimer's disease. HDAC inhibition may induce the expression of anti-mitotic and anti-apoptotic genes, such as p21 and HSP-70, which facilitate survival. HDAC inhibitors can act on other neural cell types in the central nervous system, such as reactive astrocytes and microglia, to reduce inflammation and secondary damage during neuronal injury or disease. HDAC inhibition is a promising therapeutic approach for the treatment of a range of central nervous system disorders (Langley B. et al., 2005, Current Drug Targets—CNS & Neurological Disorders, 4: 41-50).

It is known that HDAC inhibitors, with different specificities, for example SAHA and ITF2357 (Leoni et al, PNAS. 2002, 99, 2995-3000 and Mol. Med. 2005, 11, 1-15) can non-specifically reduce the levels of proinflammatory cytokines and have been demonstrated to have anti-inflammatory activity in vivo (Wang et al, Nature Rev. Drug Discov. 2009, 8, 969-981). SAHA, ITF2357, TSA, HC-toxin and tubacin inhibit secretion of mature IL-1β but do not affect the level of synthesis or the intracellular localization of the precursors of IL-1β (Carta et al. 2006, Blood; 108: 1618-1626.)

The present invention provides methods of treating a disease by administering a an HDAC6 inhibitor, that modulates specifically the extracellular level of a group of cytokines or a subset thereof.

### BRIEF SUMMARY OF THE INVENTION

The invention relates to methods of treating a subject with a disease, comprising: identifying a subject in need of treatment; administering to the subject an HDAC6 inhibitor; determining the extracellular level of a group of cytokines; wherein following the administration, there is a modulation of the extracellular level of a subset of cytokines of the group, thereby treating the disease.

The invention also relates to a method of treating a subject with a disease, comprising administering to the subject an HDAC6 inhibitor that is identified as capable of modulating the extracellular level of a subset of a group of cytokines; determining the extracellular level of the group of cytokines; wherein following the administration, there is a modulation of the extracellular level of the subset of cytokines, thereby treating the disease.

The invention also relates to a method of treating a subject with a disease, comprising: administering an HDAC6 inhibitor that is identified as capable of modulating the extracellular level of a subset of a group of cytokines wherein following the administration, there is a modulation of the extracellular level of a subset of cytokines, thereby treating the disease.

The invention also relates to a method of monitoring the treatment of a subject diagnosed with a disease, comprising determining the extracellular level of a group of cytokines of the subject; administering to the subject an HDAC6 inhibitor; and comparing the extracellular level of the group of cytokines of the subject before and after administration of the HDAC6 inhibitor.

In one embodiment of the methods of the invention, following administration of the HDAC6 inhibitor there is a modulation of the extracellular level of a subset of the group of cytokines thereby indicating treatment.

The invention also relates to a method of monitoring the treatment of a subject diagnosed with a disease, comprising: determining the extracellular level of a group of cytokines of the subject; administering to the subject an HDAC6 inhibitor; and comparing the extracellular level of the group of cytokines of the subject with the extracellular level of the group of cytokines of a control subject that is not diagnosed with the disease.

In one embodiment, following administration of the HDAC6 inhibitor there is a modulation of the extracellular level of a subset of the group of cytokines of the subject diagnosed with the disease as compared to the control subject thereby indicating treatment.

The invention also relates to a method of treating a subject with a disease, comprising: administering to the subject an HDAC6 inhibitor; and determining the extracellular level of a group of cytokines; wherein following the administration, there is a modulation of the extracellular level of a subset of the group of cytokines, thereby treating the disease.

In one embodiment modulation comprises a decrease in the extracellular level of at least one cytokine of the group.

In another embodiment modulation comprises a decrease in the extracellular level of at least one cytokine of the group and an increase in the extracellular level of at least one cytokine of the group.

In another embodiment modulation comprises a decrease in the extracellular level of at least one cytokine of the group, and wherein an increased extracellular level of the at least one cytokine is associated with a disease.

In another embodiment the extracellular level of at least one cytokine of the group is not modulated.

The invention also provides for a method of treating a subject with a disease, comprising: administering an HDAC6 inhibitor that is identified as capable of modulating the extracellular level of a subset of a group of cytokines, thereby treating the disease.

In one embodiment modulation comprises a decrease in the extracellular level of at least one cytokine of the group.

In another embodiment modulation comprises a decrease in the extracellular level of at least one cytokine of the group and an increase in the extracellular level of at least one cytokine of the group.

In another embodiment modulation comprises a decrease in the extracellular level of at least one cytokine of the group, and wherein an increased extracellular level of the at least one cytokine is associated with a disease.

In another embodiment the extracellular level of at least one cytokine of the group is not modulated.

The invention also provides a method of modulating the extracellular level of a subset of a group of cytokines, comprising contacting a cell with an inhibitor of HDAC6; and determining the extracellular level of the group of cytokines, wherein the extracellular level of a subset of the group of cytokines is modulated following the contacting.

The invention also provides a method of designing a treatment protocol for a subject diagnosed with a disease, comprising: determining the extracellular level of a group of cytokines of the subject diagnosed with a disease; and comparing the extracellular level of the group of cytokines of the subject with the extracellular level of the group of cytokines of a control subject that does not have the disease; wherein a modulation in the extracellular level of a subset of the group of cytokines in the subject as compared to the control indicates that an HDAC6 inhibitor that is identified as capable of modulating the extracellular level of a subset of the group of cytokines should be administered to the subject; and wherein no modulation in the extracellular level of a subset of the group of cytokines in the subject as compared to the control indicates that an HDAC6 inhibitor that is identified as capable of modulating the extracellular level of a subset of the group of cytokines should not be administered to the subject.

In one embodiment at least one side effect selected from the group consisting of nausea, vomiting, diarrhea, fatigue, QT/QTc prolongation, torsades de point, cardiac arrhythmias, myelosuppression, depressed blood cell count, and thrombocytopenia, lymphopenia, neutropeniais is not detected.

In another embodiment a second therapeutic agent is administered to the subject with the HDAC6 inhibitor.

In another embodiment the group of cytokines comprises at least one of the cytokines recited in any one of Tables 2-9.

In another embodiment the group of cytokines comprises at least one of the cytokines of any one of Tables, 3, 5, 6, 7, 8 and 9 and wherein the group of cytokines also comprises at least one of the cytokines of Table 4.

In another embodiment the group of cytokines comprises at least one of the cytokines in any one of Tables 5, 6, 7, 8, and 9.

In another embodiment the group of cytokines comprises TNF-α.

In another embodiment the disease is selected from the group consisting of: rheumatoid arthritis, multiple sclerosis, inflammatory bowel disease, psoriasis, and systemic lupus erythematosis.

In another embodiment the HDAC6 inhibitor is specific for HDAC6.

In another embodiment the HDAC6 inhibitor is administered in a therapeutically effective amount or a pharmaceutically acceptable salt or prodrug thereof, or a pharmaceutical composition comprising a therapeutically effective amount or a pharmaceutically acceptable salt or prodrug thereof, to the subject, thereby treating the disease.

In another embodiment the methods further comprise obtaining the HDAC6 inhibitor or the pharmaceutically acceptable salt or prodrug thereof.

In another embodiment the subject is a mammal.

In another embodiment the subject is a human.

In another embodiment the therapeutically effective amount is in the range of 1 mg to 5 gm.

In another embodiment the therapeutically effective amount of the HDAC6 inhibitor is administered by topical application, intravenous drip or injection, subcutaneous, intramuscular, intraperitoneal, intracranial and spinal injection, ingestion via oral route, inhalation, trans-epithelial diffusion or an implantable, time-release drug delivery device.

In another embodiment the results of the determining step is reported to the subject or a health care professional.

The invention also provides for a packaged pharmaceutical comprising an HDAC inhibitor or a pharmaceutically acceptable salt or prodrug thereof which, upon administration to a subject, modulates the extracellular level of a subset of a group of cytokines.

The invention also provides for a packaged pharmaceutical comprising: (a) an HDAC inhibitor or a pharmaceutically acceptable salt or prodrug thereof and (b) associated instructions for using the HDAC inhibitor to treat a disease associated with an increase in the extracellular level of a group of cytokines.

In one embodiment the HDAC inhibitor is present as a pharmaceutical composition comprising a therapeutically effective amount of the HDAC inhibitor or a pharmaceutically acceptable salt or prodrug thereof and a pharmaceutically acceptable carrier in the packaged pharmaceutical.

In another embodiment, the packaged pharmaceutical further comprises a step of identifying a subject in need of the pharmaceutical.

In another embodiment, the packaged pharmaceutical further comprises a step of identifying the HDAC inhibitor as capable of modulating the extracellular level of a subset of a group of cytokines associated with the disease.

In another embodiment of the methods of the invention the HDAC6 inhibitor is identified as having an IC50<10 μM.

The invention also provides for a method of screening for an inhibitor of HDAC6 comprising the steps of: determining the extracellular levels of a group of cytokines of a cell; contacting the cell with a compound; comparing the extracellular level of the group of cytokines before and after the contacting step; and wherein a compound that modulates the extracellular level of a subset of the group of cytokines is identified as an inhibitor of HDAC6 inhibitor.

In one embodiment, the modulation of the extracellular level of a cytokine is associated with an increase or decrease in the level of cytokine mRNA.

In another embodiment, the modulation of the extracellular level of a cytokine is associated with an increase or decrease in the level of cytokine protein.

In one embodiment, the extracellular level of a group of cytokines comprises the level of a group of cytokines in the plasma of said subject.

In another embodiment, the extracellular level of a group of cytokines is determined by measuring the level of a group of cytokines in the plasma of said subject.

Other extracellular sources include but are not limited to, for example, inflammatory exudates from tissues, cerebrospinal fluid, synovial fluid, peritoneal fluid, fluid from pulmonary edema, pericardial effusion, whole blood and serum.

### BRIEF DESCRIPTION OF THE DRAWINGS

FIG. 1 presents the effect of HDAC6 inhibitors on the LPS induced cytokine release from PBMCs (FIG. 1A-GM-CSF; FIG. 1B-IL-1β; FIG. 1C-IL-6; FIG. 1D IL-7; FIG. 1E-IL-8; FIG. 1F-IL-10; FIG. 1G-MCP-1; and FIG. 1H-TNF-α).

FIG. 2 presents the effect of HDAC6 inhibitors on LPS-induced TNF-α release from human monocyte macrophages.

FIG. 3 presents a western blot demonstrating tubulin acetylation in human monocyte derived macrophages following overnight treatment with 10 ng/ml LPS and an HDAC inhibitor.

FIG. 4 presents a graph showing inhibition of TNFα protein and mRNA in LPS stimulated RAW264.7 mouse macrophages by HDAC inhibitor ACY-738.

FIG. 5 presents inhibition by HDAC6 selective compounds of TNF-α in plasma of mice challenged with LPS: (a) compounds were administered by IP injection 4 hours prior to LPS challenge, n=8. (b) compounds were administered by either oral gavage or IP injection two hours prior to LPS challenge.

FIG. 6 presents the effect of HDAC inhibitors on the development of collagen induced arthritis in mice.

FIG. 7 presents the effect of HDAC6 inhibitors on the development of collagen induced arthritis in rats.

### DETAILED DESCRIPTION OF THE INVENTION

The invention provides for methods of treating a subject diagnosed with a disease associated with a modulation in the extracellular level of a particular subset of cytokines. In particular embodiments of the invention, the methods include an initial step of identifying a patient in need of treatment for a disease that is associated with a modulation in the extracellular level of a particular subset of cytokines. In certain embodiments, the methods of the invention include an initial step of identifying a compound capable of modulating the extracellular level of a particular subset of cytokines associated with a particular disease. The invention provides for compounds that are HDAC inhibitors, including HDAC6 inhibitors, as well as pharmaceutical compositions comprising HDAC inhibitors, that have all of the functional attributes of the compounds of the methods of the invention.

### DEFINITIONS

The term “group of cytokines” as used herein refers to at least one cytokine, interleukin, chemokine or growth factor, for example 1, 2, 3, 4, 5, 6, 7, 8, 9, 10, 15, 20 or more. In one embodiment, a “group of cytokines” refers to at least two cytokines, for example, 2, 3, 4, 5, 6, 7, 8, 9, 10, 15, 20 or more. As used herein, a “group of cytokines” also refers to at least two cytokines wherein the extracellular level of at least one of the cytokines of the group is decreased in the presence of an HDAC inhibitor, for example, an HDAC6 inhibitor, including but not limited to the cytokines presented in Tables 2-9 and in the section entitled “Cytokines”. A “group of cytokines” also refers to at least two cytokines wherein the extracellular level of at least one cytokine of the group is decreased in the presence of an HDAC inhibitor, for example, an HDAC6 inhibitor and wherein the extracellular level of at least one cytokine of the group is increased in the presence of an HDAC inhibitor, for example, an HDAC6 inhibitor. A “group of cytokines” also refers to at least two cytokines wherein the extracellular level of at least one of the cytokines of the group is decreased by an HDAC inhibitor, for example, an HDAC6 inhibitor and wherein the extracellular level of at least one cytokine of the group is not increased or decreased in the presence of an HDAC inhibitor, for example, an HDAC6 inhibitor. A cytokine that does not exhibit a decrease in extracellular level in the presence of an HDAC inhibitor, for example, an HDAC6 inhibitor includes but is not limited to any one of the cytokines presented in Table 4.

As used herein, the term “cytokine” includes but is not limited to any one of the cytokines presented in the section entitled “Cytokines” and in Tables 2-9.

As defined herein, a cytokine according to the invention includes a cytokine that demonstrates a decrease in extracellular level in response to an HDAC inhibitor, for example, an HDAC6 inhibitor. A cytokine according to the invention also includes a cytokine that does not demonstrate an increase or a decrease in extracellular level in response to an HDAC inhibitor, for example, an HDAC6 inhibitor. As defined herein, a cytokine according to the invention also includes a cytokine that demonstrates an increase in extracellular level in response to an HDAC inhibitor, for example, an HDAC6 inhibitor.

“Treatment”, or “treating” as used herein, is defined as the application or administration of an HDAC inhibitor, for example, an HDAC6 inhibitor as defined herein, to a subject or patient, or application or administration of an HDAC inhibitor, for example, an HDAC6 inhibitor to an isolated tissue or cell line from a subject or patient, who has a disease or disorder that is associated with an increased extracellular level of a group of cytokines, with the purpose to cure, heal, alleviate, relieve, alter, remedy, ameliorate, improve or affect the disease or disorder, or symptoms of the disease or disorder. The term “treatment” or “treating” is also used herein in the context of administering agents prophylactically. The term “effective dose” or “effective amount” or “effective dosage” or “therapeutic dosage” is defined as an amount sufficient to achieve or at least partially achieve the desired effect. The terms “therapeutically effective dose” and “therapeutically effective amount” are defined as an amount sufficient to cure or at least partially arrest the disease and its complications in a patient already suffering from the disease.

As used herein, “patient” or “subject” refers to a mammal that is diagnosed with a disease associated with an increase in the extracellular level of a group of cytokines.

The term “patient” or “subject” includes human and other mammalian subjects that receive either prophylactic or therapeutic treatment.

As used herein, “mammal” refers to any mammal including but not limited to human, mouse, rat, sheep, monkey, goat, rabbit, hamster, horse, cow or pig.

A “non-human mammal”, as used herein, refers to any mammal that is not a human.

As used herein, “treating” a disease refers to preventing the onset of disease and/or reducing, delaying, or eliminating disease symptoms, such as an increase in the extracellular level of a group of cytokines. By “treating” is meant restoring the patient or subject to the basal state as defined herein, and/or to prevent a disease in a subject at risk thereof. Alternatively, “treating” means arresting or otherwise ameliorating symptoms of a disease.

As used herein, “basal state” refers to the extracellular level of a group of cytokines of an individual who is not susceptible to a disease and who has no symptoms of a disease.

As used herein, the term “disease” includes any one or more of the following autoimmune diseases or disorders: diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, primary biliary cirrhosis, uveitis posterior, graft versus host disease (GVHD) and interstitial lung fibrosis.

In another embodiment, disease refers to any one of Wilson's disease, spinocerebellar ataxia, prion disease, Parkinson's disease, Huntington's disease, amytrophic lateral sclerosis, amyloidosis, Alzheimer's disease, Alexander's disease, alcoholic liver disease, cystic fibrosis, Pick's Disease, spinal muscular dystrophy or Lewy body dementia.

“Disease” also includes any one of rheumatoid spondylitis; post ischemic perfusion injury; inflammatory bowel disease; chronic inflammatory pulmonary disease, eczema, asthma, ischemia/reperfusion injury, acute respiratory distress syndrome, infectious arthritis, progressive chronic arthritis, deforming arthritis, traumatic arthritis, gouty arthritis, Reiter's syndrome, acute synovitis and spondylitis, glomerulonephritis, hemolytic anemia, aplastic anemia, neutropenia, host versus graft disease, allograft rejection, chronic thyroiditis, Graves' disease, primary binary cirrhosis, contact dermatitis, skin sunburns, chronic renal insufficiency, Guillain-Barre syndrome, uveitis, otitis media, periodontal disease, pulmonary interstitial fibrosis, bronchitis, rhinitis, sinusitis, pneumoconiosis, pulmonary insufficiency syndrome, pulmonary emphysema, pulmonary fibrosis, silicosis, or chronic inflammatory pulmonary disease.

“Disease” also refers to any one of cancer, tumor growth, cancer of the colon, breast, bone, brain and others (e.g., osteosarcoma, neuroblastoma, colon adenocarcinoma), chronic myelogenous leukemia (CML), acute myeloid leukemia (AML), acute promyelocytic leukemia (APL), cardiac cancer (e.g., sarcoma, myxoma, rhabdomyoma, fibroma, lipoma and teratoma); lung cancer (e.g., bronchogenic carcinoma, alveolar carcinoma, bronchial adenoma, sarcoma, lymphoma, chondromatous hamartoma, mesothelioma); various gastrointestinal cancer (e.g., cancers of esophagus, stomach, pancreas, small bowel, and large bowel); genitourinary tract cancer (e.g., kidney, bladder and urethra, prostate, testis; liver cancer (e.g., hepatoma, cholangiocarcinoma, hepatoblastoma, angiosarcoma, hepatocellular adenoma, hemangioma); bone cancer (e.g., osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma and giant cell tumors); cancers of the nervous system (e.g., of the skull, meninges, brain, and spinal cord); gynecological cancers (e.g., uterus, cervix, ovaries, vulva, vagina); hematologic cancer (e.g., cancers relating to blood, Hodgkin's disease, non-Hodgkin's lymphoma); skin cancer (e.g., malignant melanoma, basal cell carcinoma, squamous cell carcinoma, Karposi's sarcoma, moles dysplastic nevi, lipoma, angioma, dermatofibroma, keloids, psoriasis); and cancers of the adrenal glands (e.g., neuroblastoma).

In one embodiment, a disease according to the invention is associated with an increase in the extracellular level of at least one cytokine of the invention.

As used herein, “diagnosing” or “identifying a patient or subject having” refers to a process of determining if an individual is afflicted with a disease or ailment, for example a disease associated with an increase in the extracellular level of a group of cytokines as defined herein. To diagnose a disease associated with an increase in the extracellular level of a group of cytokines the level of one or more cytokines of the group is measured by methods known in the art including but not limited to Western blot analysis and ELISA. Additional methods useful according to the invention include but are not limited to bead based multiplex immunoassay, quantitative reverse transcriptase PCR, flow cytometry, fluorescence polarization, fluorescence resonance energy transfer, amplified luminescent proximity homogenous assay and surface plasmon resonance assay.

As used herein, “modulate” or “modulation” refers to increase or decrease, or an increase or a decrease, for example an increase or decrease in the extracellular level of a group of cytokines.

As used herein, “decrease” means that the extracellular level of a group of cytokines is 1, 2, 3, 4, 5, 10, 15, 20, 25, 30, 35, 40, 45, 50, 100, 500, 1000 or 10.000-fold less after administration of an HDAC6 inhibitor of the invention as compared to before administration of an HDAC6 inhibitor of the invention.

As used herein, “decrease” also means that the extracellular level of a group of cytokines is 1, 5, 10, 15, 20, 25, 30, 35, 40, 45, 50, 55, 60, 65, 70, 75, 80, 85, 90, 95, 99 or 100% less after administration of an HDAC inhibitor, for example, an HDAC6 inhibitor of the invention as compared to before administration of an HDAC inhibitor, for example, an HDAC6 inhibitor of the invention.

As used herein, “decrease” means that the extracellular level of a group of cytokines associated with a disease is decreased in a subject diagnosed with the disease. According to this embodiment, the disease is associated with an increase in the extracellular level of a group of cytokines. Further according to this embodiment, the decrease in the extracellular level of a group of cytokines is decreased after administration of an HDAC inhibitor, for example, an HDAC6 inhibitor of the invention such that the level of the group of cytokines is equivalent to the extracellular level of a group of cytokines associated with a disease in a control subject that does not have the disease.

As used herein “increased” as it refers to the extracellular level of a group of cytokines associated with a disease, means that the extracellular level of a group of cytokines of the invention is 1, 2, 3, 4, 5, 10, 15, 20, 25, 30, 35, 40, 45, 50, 100, 500, 1000 or 10.000-fold or more greater in a patient diagnosed with the disease as compared to a control subject that is not diagnosed with the disease.

As used herein “increased” as it refers to the extracellular level of a group of cytokines associated with a disease, as defined herein, means that the extracellular level of a group of cytokines of the invention is 1, 5, 10, 15, 20, 25, 30, 35, 40, 45, 50, 55, 60, 65, 70, 75, 80, 85, 90, 95, 99 or 100% greater in a patient diagnosed with the disease as compared to a control subject that is not diagnosed with the disease.

As used herein, “increase” means that the extracellular level of a group of cytokines is 1, 2, 3, 4, 5, 10, 15, 20, 25, 30, 35, 40, 45, 50, 100, 500, 1000 or 10.000-fold more after administration of an HDAC inhibitor, for example, an HDAC6 inhibitor of the invention as compared to before administration of an HDAC inhibitor, for example, an HDAC6 inhibitor of the invention.

As used herein, “increase” also means that the extracellular level of a group of cytokines is 1, 5, 10, 15, 20, 25, 30, 35, 40, 45, 50, 55, 60, 65, 70, 75, 80, 85, 90, 95, 99 or 100% more after administration of an HDAC inhibitor, for example, an HDAC6 inhibitor of the invention as compared to before administration of an HDAC inhibitor, for example, an HDAC6 inhibitor of the invention.

The invention provides for a decrease in the extracellular level of a group of cytokines, wherein the extracellular level of at least a first cytokine is decreased by a certain increment and wherein the extracellular level of at least a second cytokine is decreased by an increment that is larger or smaller than the increment by which the extracellular level of the first cytokine is decreased.

The invention also provides for a decrease in the extracellular level of a group of cytokines, wherein the extracellular level of at least a first cytokine is decreased by a certain increment and wherein the extracellular level of at least a second cytokine does not change.

The invention provides for a decrease in the extracellular level of a group of cytokines, wherein the extracellular level of at least a first cytokine is decreased by a certain increment and wherein the extracellular level of at least a second cytokine is increased. As used herein, “extracellular” means outside of the cell.

As used herein, “intracellular” means inside of the cell.

As used herein “extracellular level” means the level outside of the cell.

As used herein, “trafficking” means the movement of proteins within the cell, the transport of proteins from an intracellular location to an extracellular location, and the route proteins take from an intracellular location to an extracellular location.

The invention provides for methods of determining the extracellular level of a cytokine in vitro or in vivo, for example, in a patient, including but not limited to Western blot analysis and ELISA analysis. Additional methods useful according to the invention include but are not limited to bead based multiplex immunoassay, quantitative reverse transcriptase PCR, flow cytometry, fluorescence polarization, fluorescence resonance energy transfer, amplified luminescent proximity homogenous assay and surface plasmon resonance assay.

A method of “administration” useful according to the invention includes but is not limited to subcutaneous, intramuscular, intraperitoneal, intracranial and spinal injection, ingestion via the oral route, inhalation, trans-epithelial diffusion (such as via a drug-impregnated, adhesive patch), by the use of an implantable, time-release drug delivery device, which may comprise a reservoir of exogenously-produced agent or may, instead, comprise cells that produce and secrete the therapeutic agent or topical application or administration directly to a blood vessel, including artery, vein or capillary, intravenous drip or injection. Additional methods of administration are provided herein below in the section entitled “Dosage and Administration.”

As used herein, the terms “histone deacetylase”, “HDAC”, “histone deacetylase isoform”, “HDAC isoform” and similar terms are intended to refer to any one of a family of enzymes that remove acetyl groups from the epsilon-amino groups of lysine residues at the N-terminus of a histone and non-histone proteins. Histone deacetylase isoforms include class I and class II enzymes. Specific HDACs include without limitation, HDAC1, HDAC2, HDAC3, HDAC4, HDAC5, HDAC6, HDAC7, HDAC8, HDAC9 and HDAC10. In one embodiment, histone deaceytylase is HDAC6.

As used herein, an “HDAC inhibitor” means a compound that inhibits the deacetylase activity of an HDAC by 1, 2, 3, 4, 5, 10, 15, 20, 25, 30, 35, 40, 45, 50, 100, 500, 1000 or 10,000-fold as compared to an HDAC in the absence of the inhibitor.

An “HDAC inhibitor” also refers to a compound that inhibits the deacetylase activity of an HDAC by 1, 5, 10, 15, 20, 25, 30, 35, 40, 45, 50, 55, 60, 65, 70, 75, 80, 85, 90, 95, 99 or 100% as compared to the activity of an HDAC in the absence of the inhibitor.

As used herein, an HDAC6 inhibitor” means a compound that inhibits the deacetylase activity of an HDAC6 by 1, 2, 3, 4, 5, 10, 15, 20, 25, 30, 35, 40, 45, 50, 100, 500, 1000 or 10,000-fold as compared to an HDAC6 in the absence of the inhibitor

An “HDAC6 Inhibitor” also refers to a compound that inhibits the deacetylase activity of HDAC6 by 1, 5, 10, 15, 20, 25, 30, 35, 40, 45, 50, 55, 60, 65, 70, 75, 80, 85, 90, 95, 99 or 100% as compared to the activity of HDAC6 in the absence of the inhibitor.

“HDAC inhibitors” useful according to the invention include but are not limited to ACY-63, ACY-216, ACY-251 and ACY-257, hydroxamic acid based HDAC inhibitors, Suberoylanilide hydroxamic acid (SAHA) and its derivatives, NVP-LAQ824, Trichostatin A, Scriptaid, m-Carboxycinnamic acid bishydroxamic acid (CBHA), ABHA, Pyroxamide, Propenamides, Oxamflatin, 6-(3-Chlorophenylureido)caproic hydroxamic acid (3-CI-UCHA), A-161906, jnj16241199, tubacin and tubacin analogs, siRNA, short chain fatty acid HDAC inhibitors, butyrate, phenylbutyrate, valproate, hydroxamic acid, trichostatins, epoxyketone-containing cyclic tetrapeptides, HC-toxin, Chlamydocin, Diheteropeptide, WF-3161, Cyl-1, Cyl-2, non-epoxyketone-containing cyclic tetrapeptides, Apicidin, cyclic-hydroxamic-acid-containing peptides (CHAPS), benzamides and benzamide analogs, CI-994, deprudecin, organosulfur compounds, Tubastatin A, ISOX, LBH-589 and FK-228 and any combination thereof.

“HDAC6” inhibitors useful according to the invention include but are not limited to ACY-63, ACY-216, ACY-251, ACY-257, tubacin, tubacin analogs, tubastatin, ISOX, HDAC6-specific siRNAs, SAHA, trichostatin, Scriptaid, LBH-589 and PXD-101.

“HDAC6-specific” inhibitors useful according to the invention include but are not limited to ACY-63, ACY-216, ACY-251, ACY-257, tubacin, tubacin analogs, tubastatin, ISOX and HDAC6-specific siRNAs.

An “HDAC6 specific inhibitor” means a compound that inhibits the deacetylase activity of HDAC6 by 1, 5, 10, 15, 20, 25, 30, 35, 40, 45, 50, 55, 60, 65, 70, 75, 80, 85, 90, 95, 99 or 100% as compared to the activity of a second HDAC that is not HDAC6.

An “HDAC6 specific inhibitor” means a compound that selectively inhibits the deacetylase activity of HDAC6 by 2, 5, 10, 15, 20, 25, 30, 35, 40, 45, 50, 100, 500, 1000 or 10.000-fold compared to the inhibitory activity of the same compound against Class I (HDAC1, 2 and 3), Class II (HDAC4, 5, 7, 8, 9 and 10) and Class IV (HDAC11) HDACs (see for example Table 1)

The invention also provides for HDAC inhibitors that decrease the extracellular level of a subset of cytokines (including but not limited to the cytokines presented in Table 3).

The invention also provides for HDAC inhibitors that neither increase nor decrease the extracellular level of a subset of cytokines (including but not limited to the cytokines presented in Table 4).

The invention also provides for HDAC inhibitors that increase the extracellular level of a subset of cytokines.

The invention also provides for HDAC6 inhibitors that decrease the extracellular level of a subset of cytokines (including but not limited to the cytokines presented in Table 3).

The invention also provides for HDAC6 inhibitors that neither increase nor decrease the extracellular level of a subset of cytokines (including but not limited to the cytokines presented in Table 4).

The invention also provides for HDAC6 inhibitors that increase the extracellular level of a subset of cytokines.

By way of non-limiting example, useful agents that inhibit one or more deacetylase, for example, histone deacetylase, isoforms, include compounds including but not limited to small molecule inhibitors and antisense oligonucleotides.

As used herein, the term “small molecule” refers to a non-peptidic, non-oligomeric organic compound either synthesized in the laboratory or found in nature. Small molecules, as used herein, can refer to compounds that are “natural product-like”. However, the term “small molecule” is not limited to “natural product-like” compounds. Rather, a small molecule is typically characterized in that it contains several carbon-carbon bonds, and has a molecular weight of less than 1500, although this characterization is not intended to be limiting for the purposes of the present invention. Examples of “small molecules” that occur in nature include, but are not limited to, taxol, dynemicin, and rapamycin. In certain other preferred embodiments, natural-product-like small molecules are utilized.

An “HDAC” inhibitor useful according to the invention has an 1050 or “potency” against a cytokine wherein the extracellular level of the cytokine is modulated by the HDAC inhibitor is in the range of less than 10 μM and greater than 1 pM. In another embodiment, an “HDAC” inhibitor useful according to the invention has an 1050 or “potency” against a cytokine wherein the extracellular level of the cytokine is modulated by the HDAC inhibitor is in the range of less than 100 nM and greater than 100 pM. In another embodiment, an “HDAC” inhibitor useful according to the invention has an 1050 or “potency” against a cytokine wherein the extracellular level of the cytokine is modulated by the HDAC inhibitor in the range of less than 100 nM and greater than 100 pM.

In one embodiment of the invention, the 1050 against a cytokine that is not modulated by an HDAC inhibitor of the invention is 10,000 fold, 100-fold, 100-fold, 10-fold or 3-fold less than the 1050 against a target.

A “therapeutically effective amount” of an HDAC inhibitor according to the invention is in the range of 1 mg-5 gm per subject. In another embodiment, a “therapeutically effective amount” of an HDAC inhibitor according to the invention is in the range of 10 mg-1 gm per subject. In another embodiment, a “therapeutically effective amount” of an HDAC inhibitor according to the invention is in the range of 1 mg-1 gm per subject.

The invention provides for methods for measuring deacetylase activity, for example, histone deacetylase activity, including but not limited to measuring acetylation of tubulin or histone in treated cells by western blot and measuring the activity of recombinant enzymes on synthetic substrates in kinetic mode as described in the examples. Deacetylase activity can also be measured in an endpoint fluorescent assay using recombinant enzymes. Acetylation of tubulin, histone or other HDAC substrates can also be measured in cells by fluorescence microscopy, flow cytometry or ELISA of whole cell lysates

An HDAC inhibitor, for example, an HDAC6 inhibitor, useful according to the invention “modulates” the extracellular level of a group of cytokines, as defined herein.

As used herein “monitoring the treatment” means determining whether, following treatment of a subject, for example, a subject diagnosed with a disease associated with an increased extracellular level of a group of cytokines, the subject has been treated such that the symptoms of the disease are arrested or otherwise ameliorated and/or the disease and/or its attendant symptoms are alleviated or abated.

As used herein, “control subject” means a subject that has not been diagnosed with a disease and/or does not exhibit any detectable symptoms associated with the disease.

As used herein, the term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters of the compounds formed by the process of the present invention which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds formed by the process of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to afford any compound delineated by the formulae of the instant invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

As used herein “side effects” includes but is not limited to any one of nausea, vomiting, diarrhea, fatigue, QT/QTc prolongation, torsades de point, cardiac arrhythmias, myelosuppression, depressed blood cell count, and consequences thereof, e.g., thrombocytopenia, lymphopenia, neutropenia.

I. Histone Deacetylases

Nucleosomes, the primary scaffold of chromatin folding, are dynamic macromolecular structures, influencing chromatin solution conformations (Workman and Kingston, 1998). The nucleosome core is made up of histone proteins, H2A, HB, H3 and H4. Histone acetylation causes nucleosomes and nucleosomal arrangements to behave with altered biophysical properties. The balance between activities of histone acetyl transferases (HAT) and deacetylases (HDAC) determines the level of histone acetylation. Acetylated histones cause relaxation of chromatin and activation of gene transcription, whereas deacetylated chromatin generally is transcriptionally inactive.

Eleven different HDACs have been cloned from vertebrate organisms (HDAC 1, HDAC2 and HDAC3 (termed Class I human HDACs), and HDAC8 (Van den Wyngaert et al., 2000). Class II HDACs, HDAC4, HDAC5, HDAC6, HDAC7, HDAC9, and HDAC10 (Kao et al., 2000) have been cloned and identified (Grozinger et al., 1999; Zhou et al. 2001; Tong et al., 2002). Additionally, HDAC 11 has been identified (Gao et al., 2002) and has properties of both class I and II HDACS.

In preferred embodiments, an HDAC inhibitor useful in the invention has one or more of the following properties: an HDAC inhibitor is capable of modulating a subset of cytokines of a group according to the invention, is capable of inhibiting at least one histone deacetylase; the compound is capable of inhibiting HDAC6; the compound is a selective HDAC6 inhibitor; the compound binds to the poly-ubiquitin binding domain of HDAC6; is capable of inducing apoptosis in cancer cells (especially multiple myeloma cells, non-Hodgkin's lymphoma (NML) cells, breast cancer cells, acute myelogenous leukemia (AML) cells); and/or is capable of inhibiting aggresome formation.

In certain preferred embodiments, an HDAC inhibitor of the invention comprises a metal binding moiety, preferably a zinc-binding moiety such as a hydroxamate. As noted above, certain hydroxamates are potent inhibitors of HDAC6 activity; without wishing to be bound by theory, it is believed that the potency of these hydroxamates is due, at least in part, to the ability of the compounds to bind zinc. In preferred embodiments, a compound of the invention includes at least one portion or region which can confer selectivity for a biological target implicated in the aggresome pathway, e.g., a biological target having tubulin deacetylase (TDAC) or HDAC activity, e.g., HDAC6. Thus, in certain preferred embodiments, an HDAC inhibitor of the invention includes a zinc-binding moiety spaced from other portions of the molecule which are responsible for binding to the biological target.

II. HDAC Inhibitors

HDACs can be inhibited through a variety of different mechanisms—proteins, peptides, and nucleic acids (including antisense and RNAi molecules).

Also contemplated are small molecule inhibitors. Perhaps the most widely known small molecule inhibitor of HDAC function is Trichostatin A, a hydroxamic acid. It has been shown to induce hyperacetylation and cause reversion of ras transformed cells to normal morphology (Taunton et al., 1996) and induces immunsuppression in a mouse model (Takahashi et al. 1996). It is commercially available from BIOMOL Research Labs, Inc., Plymouth Meeting, Pa.

“HDAC inhibitors” useful according to the invention include but are not limited to ACY-63, ACY-216, ACY-251 and ACY-257, hydroxamic acid based HDAC inhibitors, Suberoylanilide hydroxamic acid (SAHA) and its derivatives, NVP-LAQ824, Trichostatin A, Scriptaid, m-Carboxycinnamic acid bishydroxamic acid (CBHA), ABHA, Pyroxamide, Propenamides, Oxamflatin, 6-(3-Chlorophenylureido)caproic hydroxamic acid (3-CI-UCHA), A-161906, jnj16241199, tubacin and tubacin analogs, siRNA, short chain fatty acid HDAC inhibitors, butyrate, phenylbutyrate, valproate, hydroxamic acid, trichostatins, epoxyketone-containing cyclic tetrapeptides, HC-toxin, Chlamydocin, Diheteropeptide, WF-3161, Cyl-1, Cyl-2, non-epoxyketone-containing cyclic tetrapeptides, Apicidin, cyclic-hydroxamic-acid-containing peptides (CHAPS), benzamides and benzamide analogs, CI-994, deprudecin, organosulfur compounds, Tubastatin A, ISOX, LBH-589 and FK-228 and any combination thereof.

“HDAC6” inhibitors useful according to the invention include but are not limited to ACY-63, ACY-216, ACY-251, ACY-257, tubacin, tubacin analogs, tubastatin, ISOX, HDAC6-specific siRNAs, SAHA, trichostatin, Scriptaid, LBH-589 and PXD-101.

“HDAC6-specific” inhibitors useful according to the invention include but are not limited to ACY-63, ACY-216, ACY-251, ACY-257, tubacin, tubacin analogs, tubastatin, ISOX and HDAC6-specific siRNAs.

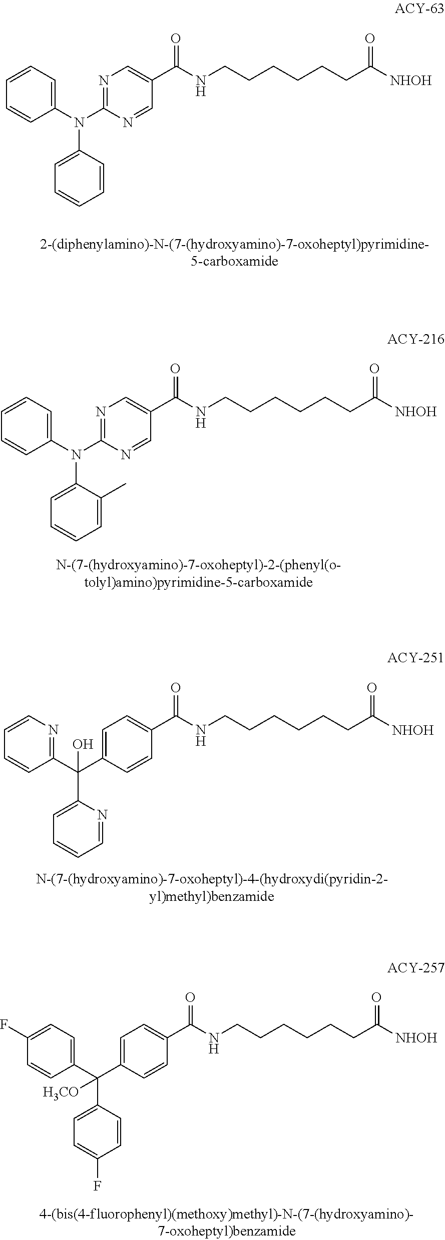

TABLE 1
Enzyme inhibition activity of compounds in nM

| Compound | HDAC1 | HDAC2 | HDAC3 | HDAC4 | HDAC5 | HDAC6 | HDAC7 | HDAC9 |
|---|---|---|---|---|---|---|---|---|
| SAHA | 40.6 | 65.7 | 40.8 | >10,000 | >10,000 | 17.7 | >10,000 | >10,000 |
| (BPS) | | | | | | | | |
| ACY-63 | 34 | 64 | 62 | 7000 | 5000 | 5.4 | 1400 | >10,000 |
| ACY-216 | 57 | 131 | 144 | >10,000 | >10,000 | 9 | >10,000 | >10,000 |
| ACY-251 | 45 | 96 | 123 | >10,000 | 5900 | 4 | >10,000 | >10,000 |
| ACY-257 | 178 | 450 | 1225 | >10,000 | 8200 | 13 | >10,000 | >10,000 |
| ACY-737 | 1268 | 1830 | 4676 | ND | ND | 6 | ND | ND |
| ACY-738 | 94 | 128 | 219 | 1600 | 882 | 2 | 1600 | 914 |
| ACY-751 | 2226 | 2971 | 4515 | ND | ND | 7 | ND | ND |
| ACY-765 | 223 | 270 | 549 | ND | ND | 3 | ND | ND |
| ACY-774 | 3206 | 4761 | 13474 | ND | ND | 9 | ND | ND |

In various embodiments, the invention provides a method further comprising co-administering to a subject an HDAC6 inhibitor in combination with a second therapeutic agent including but not limited to one or more of a chemotherapeutic agent, radiation agent, hormonal agent, biological agent or an anti-inflammatory agent to the subject. The invention also provides a method further comprising administering an HDAC6 inhibitor in combination with one or more therapeutic agents in addition to an HDAC6 inhibitor.

In a further embodiment, the anti-inflammatory agent is hormonal, steroidal anti-inflammatory agents, such as but not limited to, estradiol, conjugated estrogens (e.g., PREMARIN, PREMPRO, AND PREMPHASE), 17 beta estradiol, calcitonin-salmon, levothyroxine, dexamethasone, medroxyprogesterone, prednisone, cortisone, flunisolide, and hydrocortisone; non-steroidal anti-inflammatory agents, such as but not limited to, tramadol, fentanyl, metamizole, ketoprofen, naproxen, nabumetone, ketoralac, tromethamine, loxoprofen, ibuprofen, aspirin, and acetaminophen; disease-modifying antirheumatic agents (DMARDs), such as but not limited to, methotrexate, biologic disease-modifying anti-rheumatic agents, such as but not limited to, anti-TNF-α antibodies, such as infliximab (REMICADE™) and adalimumab (Humira™), fusion proteins containing the ligand-binding domain of TNF-α, such as etanercept (ENBREL™), and interleukin-1 (IL-1) receptor antagonist, such as anakinra (KINERET™),

In a further embodiment, the chemotherapeutic agent is tamoxifen, trastuzamab, raloxifene, doxorubicin, fluorouracil/5-fu, pamidronate disodium, anastrozole, exemestane, cyclophosphamide, epirubicin, letrozole, toremifene, fulvestrant, fluoxymester-one, trastuzumab, methotrexate, megastrol acetate, docetaxel, paclitaxel, testolactone, aziridine, vinblastine, capecitabine, goselerin acetate, zoledronic acid, taxol, vinblastine, or vincristine.

III. Cytokines

Cytokines useful according to the invention include but are not limited to any one of the cytokines presented in Tables 2-9

TABLE 2
All Cytokines

| | | | |
|---|---|---|---|
| β-NGF | IFN-γ | IL-27 | NAP-2 |
| 6Ckine/CCL2 | IL-1α | IL-28A | PDGF-AA |
| BCA-1/CXCL13 | IL-1β | IL-28B | PDGF-AB/BB |
| | IL-1ra | IL-29 | PDGF-BB |
| CTACK | IL-2 | IL-33 | RANTES |
| EGF | IL-2Rα | IP-10 | Resistin |
| ENA-78 | IL-3 | I-TAC | SCF |
| Eotaxin | IL-4 | JE/CCL2 | SCGF-β |
| Eotaxin-2/CCL24 | IL-5 | KC(CXCL1) | SDF-1α |
| Eotaxin-3/CCL26 | IL-6 | KGF | SDF-1α + β |
| EPO | IL-7 | LIF | TARC |
| Exodus-2 | IL-8 | Lymphotactin | TGF-α |
| FGF Basic | IL-9 | LIX | TGF-β1 |
| FGF-4 | IL-10 | MCP-1 | TGF-β2 |
| Flt-3L | IL-11 | MCP-2 | TGF-β3 |
| Fractalkine | IL-12 (p40) | MCP-3 | TIMP-1 |
| GCP2/CXL6 | IL-12 (p70) | MCP-4 | TL-1a |
| G-CSF | IL-13 | M-CSF | TNF-α |
| GM-CSF | IL-15 | MDC(CCL22) | TPO |
| GRO | IL-16 | MIF | TRAIL |
| GRO-α | IL-17 | MIG | TSLP |
| HCC-1/3 | IL-18 | MIP-1α | VCAM-1 |
| HGF | IL-20 | MIP-1β | VEGF |
| I-309/CCL1 | IL-21 | MIP-1D | VEGF-C |
| ICAM-1 | IL-22 | MIP-2 | VEGF-D |
| IFN-α2 | IL-23(p19/p40) | MIP-3α | |
| IFN-β | IL-25 | MIP-3β | |

TABLE 3
Cytokines affected by HDAC6 inhibition

| | |
|---|---|
| GM-CSF | IL-6 |
| IL-1β | IL12(p40) |
| MCP-1 | TNF-α |

TABLE 4
Cytokines unaffected by HDAC6 inhibition

| | |
|---|---|
| IFN-γ | IL-2 |
| IL-4 | IL-10 |
| IL-7 | IL-8 |

TABLE 5
Cytokines involved in rheumatoid arthritis

| | | | |
|---|---|---|---|
| 6Ckine/CCL2 | IL-1β | MCP-2 | IL-15 |
| EGF | IL-6 | M-CSF | IL-23 |
| Fractalkine | IL-12 (p40) | MIP-1α | IL-2 |
| G-CSF | IL-17 | RANTES | IL-4 |
| GM-CSF | IP-10 | SDF-1α | IL-13 |
| ICAM-1 | I-TAC | TARC | bFGF |
| IFN-γ | MCP-1 | TNF-α | |

TABLE 6
Cytokines involved in multiple sclerosis

| | | | |
|---|---|---|---|
| IP-10 | MIG | I-TAC | IL-6 |
| IL-1β | IL-12 | IL-23 | TGFβ |
| IL-27 | IL-17 | IFNγ | |

TABLE 7
Cytokines involved in inflammatory bowel disease

| | | | |
|---|---|---|---|
| IL-1β | IL-6 | IL-8 | IL-12 |
| IL-18 | IL-23 | IL-27 | TNF-α |
| IL-17 | IL-21 | IL-22 | IFN-γ |

TABLE 8
Cytokines involved in psoriasis

| | | | |
|---|---|---|---|
| IL-17 | IL-23 | IL-22 | IL-6 |
| IL-8 | GRO-1 | MCP-1 | TNF-α |

TABLE 9
Cytokines involved in systemic lupus erythematosis

| | | | |
|---|---|---|---|
| IL-1α | IL-1β | IFN-α | IFN-β |
| MCP-1 | MIG | IP-10 | I-TAC |

Cytokines useful according to the invention also include any one of Activin A, Adipocyte-derived leucine aminopeptidase, Adiponectin, AITRL, Alpha-taxilin, Amhiregulin, Angiopoietin-1, Angiopoietin-2, Angiopoietin-like Protein-3, Angiopoietin-like Protein-4, Apolipoprotein D, Apolipoprotein J, Apoptosis regulatory protein Siva, Artemin, BAFF, B-cell stimulating factor 3, Betacellulin, Bone Morphogenetic Protein-1, Bone Morphogenetic Protein-2, Bone Morphogenetic Protein-3, Bone Morphogenetic Protein-3b, Bone Morphogenetic Protein-4, Bone Morphogenetic Protein-5, Bone Morphogenetic Protein-6, Bone Morphogenetic Protein-7, Bone Morphogenetic Protein-8B, Bone Morphogenetic Protein-10, Bone Morphogenetic Protein-15, BRAK (CXCL14), Brain Derived Neurotrophic Factor, B-type Natriuretic Protein, Cardiotrophin-1, CD4, CD40 Ligand/TRAP, Chemokine MIP-2 gamma, Chemokine-like factor, Chorionic somatomammotropin hormone, Ciliary Neurotrophic Factor, Colony Stimulating Factor 3, Complement C5, Connective Tissue Growth Factor, Cytokine SCM-1 beta, Cytotoxic T-Lymphocyte Associated Antigen-4, Beta-Defensin-3, Ectodysplasin A, Embryonic Growth/Differentiation Factor 1, Endocrine Gland Vascular Endothelial Growth Factor, Endoglin, Eotaxin (CCL11), Epidermal Growth Factor, Epidermal Growth Factor 21, Epithelial Neutrophil-Activating Protein-78 (CXCL5), Erythropoietin alpha, Exodus-2 (CCL21), Fibroblast Growth Factor-9, Fibroblast Growth Factor-19, Fibroblast Growth Factor-21, Fibroblast Growth Factor-22, Fibroblast Growth Factor-23, Fibroblast Growth Factor acidic, Fibroblast Growth Factor basic, Flt3-Ligand, Follistatin, Fractalkine, Glial Derived Neurotrophic Factor, GM-CSF/IL-3, Glomulin, Glucose-6-phosphate isomerase, Granulins, Granulocyte Colony Stimulating Factor, Granulocyte Macrophage Colony Stimulating Factor, GRO-alpha (CXCL1), GRO-beta/MIP-2 (CXCL2), GRO-gamma (CXCL3), Growth Regulated Protein alpha, Growth and Differentiation Factor-2, Growth and Differentiation Factor-3, Growth and Differentiation Factor-5, Growth and Differentiation Factor-8, Growth and Differentiation Factor-9, Growth and Differentiation Factor-11, Growth and Differentiation Factor-15, Growth Hormone, Growth Hormone Binding Protein, HCC-1 (CCL14), Hepatocyte Growth Factor, I-309 (CCL1), IL-1F7b, IL7 nirs variant 2, Insulin, Insulin-Like Growth Factor-I, Insulin-Like Growth Factor-II, Insulin-Like Growth Factor Binding Protein-1, Insulin-Like Growth Factor Binding Protein-3, Insulin-Like Growth Factor Binding Protein-5, Insulin-Like Growth Factor Binding Protein-6, Interferon alpha, Interferon alpha1, Interferon alpha1/13, Interferon alpha1a, Interferon alpha1b, Interferon alpha2b, Interferon alpha-2, Interferon alpha-4, Interferon alpha-5, Interferon alpha-6, Interferon alpha-7, Interferon alpha-8, Interferon alpha-10, Interferon alpha-14, Interferon alpha-16, Interferon alpha-17, Interferon beta1a, Interferon beta1 b, Interferon delta-1, Interferon gamma, Interferon kappa, Interferon omega-1, Interferon Regulatory Factor-1, Interferon Regulatory Factor-3, Interleukin-1 alpha, Interleukin-1 beta, Interleukin-2, Interleukin-3, Interleukin-4, Interleukin-5, Interleukin-6, Interleukin-7, Interleukin-8 (CXCL8), Interleukin-9, Interleukin-10, Interleukin-11, Interleukin-12, Interleukin-12 beta chain, Interleukin-13, Interleukin-15, Interleukin-16, Interleukin-17A, Interleukin-17B, Interleukin-17C, Interleukin-17E, Interleukin-18, Interleukin-19, Interleukin-20, Interleukin-21, Interleukin-22, Interleukin-24, Interleukin-26, Interleukin-28A, Interleukin-28B, Interleukin-29, Interleukin-33, Interleukin-1 Receptor Antagonist Protein, IP-10 (CXCL10), I-TAC (CXCL11), Keratinocyte Growth Factor, Keratinocye Growth Factor-2, Kit Ligand, LAP2 protein, Left-right Determination Factor B, Leptin, Leukemia Inhibitory Factor, Lyphotactin, Lymphotoxin-alpha, Lymphotoxin-beta, LeukinFeron, Leukocyte Function Associated Antigen-3, Lymphotactin, Macrophage Colony Stimulating Factor, Macrophage Inflammatory Protein-1 alpha (CCL3), Macrophage Inflammatory Protein-1 beta (CCL4), Macrophage Inflammatory Protein-2 alpha, Macrophage Inflammatory Protein-2 beta, Macrophage Inflammatory Protein-3 beta (CCL19), Macrophage Inflammatory Protein-4 (CCL18), Macrophage Migration Inhibitory Factor, Melanoma Inhibitory Activity Protein, Midkine, MIG (CXCL9), Monocyte Chemotactic Protein-1/MCAF (CCL2), Monocyte Chemotactic Protein-2 (CCL8), Monocyte Chemotactic Protein-3 (CCL7), Monocyte Chemotactic Protein-4 (CCL13), Mucin-4, Multisynthease Complex Auxiliary Component p43, Myostatin, Nerve Growth Factor beta, Neuregulin-1/Heregulin-b1, Neuroglobin, Neurotrophin-3, Neutrophil Activating Protein-2 (CXCL7), Neutrophil Activating Protein-4, Nicotinamide phosphoribosyltransferase, Nodal homolog, Noggin, Oncostatin M, Osteopontin, Osteoprotegerin, P600 homolog, Periostin, Pituitary Growth Hormone-20K, Placenta Growth Factor-1, Placenta Growth Factor-2, Placental Growth Hormone-20K, Placental-Corr Growth Hormone, Platelet Basic Protein, Platelet Factor 4, Platelet Factor 4 Variant, Platelet Derived Growth Factor-A, Platelet Derived Growth Factor-AA, Platelet Derived Growth Factor-AB, Platelet Derived Growth Factor-B, Platelet Derived Growth Factor-BB, Platelet Factor-4 (CXCL4), Pleiotrophin, Prolactin, Pro-Nerve Growth Factor, Protein FAM3B, Protein FAM3C, Protein FAM3D, RANK ligand, Rantes (CCL5), Red Protein, RELM-beta, Resistin, Secreted and Transmembrane Protein 1, Secreted Ly-6/uPAR Related Protein 1, SL Cytokine, Small Inducible Cytokine A1, Small Inducible Cytokine A2, Small Inducible Cytokine A3, Small Inducible Cytokine A3-like, Small Inducible Cytokine A4, Small Inducible Cytokine A5, Small Inducible Cytokine A8, Small Inducible Cytokine A13, Small Inducible Cytokine A14, Small Inducible Cytokine A15, Small Inducible Cytokine A16, Small Inducible Cytokine A17, Small Inducible Cytokine A18, Small Inducible Cytokine A19, Small Inducible Cytokine A20, Small Inducible Cytokine A21, Stem Cell Factor A22, Small Inducible Cytokine A23, Small Inducible Cytokine A24, Small Inducible Cytokine A25, Small Inducible Cytokine A26, Small Inducible Cytokine A27, Small Inducible Cytokine A28, Small Inducible Cytokine B5, Small Inducible Cytokine B6, Small Inducible Cytokine B9, Small Inducible Cytokine B10, Small Inducible Cytokine B11, Small Inducible Cytokine B12, Small Inducible Cytokine B13, Small Inducible Cytokine B16, Stromal Cell-Derived Factor-1 alpha (CXCL12), Stromal Cell-Derived Factor-1 beta (CXCL12), Thrombopoietin, Thymidine Phosphorylase, TNF-Related Apoptosis Inducing Ligand/Apo2L, TNF Superfamily Ligand TL1A, Transforming Growth Factor beta1, Transforming Growth Factor beta3, Tumor Necrosis Factor alpha, Tumor Necrosis Factor beta, Tumor Necrosis Factor Ligand Superfamily Member 4, Tumor Necrosis Factor Ligand Superfamily Member 5, Tumor Necrosis Factor Ligand Superfamily Member 6, Tumor Necrosis Factor Ligand Superfamily Member 7, Tumor Necrosis Factor Ligand Superfamily Member 8, Tumor Necrosis Factor Ligand Superfamily Member 9, Tumor Necrosis Factor Ligand Superfamily Member 11, Tumor Necrosis Factor Ligand Superfamily Member 11B, Tumor Necrosis Factor Ligand Superfamily Member 12, Tumor Necrosis Factor Ligand Superfamily Member 13, Tumor Necrosis Factor Ligand Superfamily Member 13B, Tumor Necrosis Factor Ligand Superfamily Member 14, Tumor Necrosis Factor Ligand Superfamily Member 15, Tumor Necrosis Factor Ligand Superfamily Member 18, Uteroglobin, Uteroglobin-related Protein 2, Vascular Endothelial Growth Factor, Vascular Endothelial Growth Factor-121, Vascular Endothelial Growth Factor-164, Vascular Endothelial Growth Factor-165, Vascular Endothelial Growth Inhibitor, Vaspin, and Visfatin.

IV. Diseases

The invention provides for methods of disease treatment. The invention also provides for methods of monitoring the treatment of a subject diagnosed with a disease. The invention also provides for a method of designing a treatment protocol for a subject diagnosed with a disease.

In one embodiment, the disease of the invention is associated with a modulation, as defined herein, of the extracellular level of a group of cytokines.

As discussed above, the present invention provides methods for the treatment of various diseases.

The methods of the invention are especially effective to treat or prevent inflammatory, immune and autoimmune diseases including, but not limited to, arthritic conditions, such as, rheumatoid arthritis, osteoarthritis; juvenile arthritis, psoriatic arthritis, rheumatoid spondylitis; psoriasis; post ischemic perfusion injury; chronic inflammatory pulmonary disease, eczema, asthma, ischemia/reperfusion injury, acute respiratory distress syndrome, psoriatic arthritis, infectious arthritis, progressive chronic arthritis, deforming arthritis, traumatic arthritis, gouty arthritis, Reiter's syndrome, polychondritis, acute synovitis and spondylitis, glomerulonephritis (with or without nephrotic syndrome), autoimmune hematologic disorders (e.g. hemolytic anemia, aplastic anemia, idiopathic thrombocytopenia and neutropenia), autoimmune gastritis and autoimmune inflammatory bowel diseases (e.g. ulcerative colitis and Crohn's disease), host versus graft disease, allograft rejection, chronic thyroiditis, Graves' disease, scleroderma, diabetes (type I and type II), active hepatitis (acute and chronic), primary binary cirrhosis, myasthenia gravis, multiple sclerosis (MS), systemic lupus erythematosus, atopic dermatitis, contact dermatitis, skin sunburns, chronic renal insufficiency, Stevens-Johnson syndrome, idiopathic sprue, sarcoidosis, Guillain-Barre syndrome, uveitis, conjunctivitis, keratoconjunctivitis, otitis media, periodontal disease, pulmonary interstitial fibrosis, bronchitis, rhinitis, sinusitis, pneumoconiosis, pulmonary insufficiency syndrome, pulmonary emphysema, pulmonary fibrosis, silicosis, chronic inflammatory pulmonary disease (e.g. chronic obstructive pulmonary disease), graft versus host disease (GVHD) and other inflammatory or obstructive diseases of the airways.

In one embodiment, the HDAC6 inhibitors of the invention are useful for treating any one or more of the following autoimmune diseases or disorders, systemic lupus erythematosis, autoimmune thyroiditis, dermatitis (including atopic dermatitis and eczematous dermatitis), psoriasis, including keratoconjunctivitis sicca secondary to Sjögren's Syndrome, alopecia areata, allergic responses due to arthropod bite reactions, aphthous ulcer, iritis, conjunctivitis, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, idiopathic thrombocytopenia, polychondritis, Wegener's granulomatosis, chronic active hepatitis, lichen planus, sarcoidosis, primary biliary cirrhosis, uveitis posterior, and interstitial lung fibrosis.

Additionally, the methods of the invention may also be useful in the treatment of protozoal infections. The methods of the invention are also useful in the treatment of diseases associated with aberrant protein catabolism, for example, protein degradation disorders, disorders associated with misfolded proteins, and protein deposition disorders. In certain embodiments, the HDAC6 inhibitors of the invention are useful in the treatment of the protein deposition disorders, Wilson's disease, spinocerebellar ataxia, prion disease, Parkinson's disease, Huntington's disease, amyotrophic lateral sclerosis, spinal muscular atrophy, spinal and bulbar muscular atrophy, amyloidosis, Alzheimer's disease, Alexander's disease, alcoholic liver disease, cystic fibrosis, Pick's disease, and Lewy body dementia. In certain exemplary embodiments, the claimed methods of the invention are useful for treating disorders associated with histone deacetylation activity. In certain exemplary embodiments, the methods of the invention are useful for treating disorders associated with tubulin deacetylation activity.

Neurodegenerative diseases that can be treated or prevented include Alzheimer's disease, Parkinson's disease, cerebral ischaemia, traumatic neurodegenerative disease, Huntington's disease or chorea, senile dementia, memory disorder, vascular dementia, lesions associated with cerebral ischemia (stroke) and with cranial and medullary trauma, among others.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

Preferably, the HDAC6 inhibitors are selective inhibitors of HDAC6 and, as such, are useful in the treatment of disorders modulated by histone deacetylases. In one embodiment, the HDAC6 inhibitors of the invention are selective inhibitors of tubulin deacetylases and, as such, are useful in the treatment of disorders modulated by tubulin deacetylases.

Thus, in another aspect of the invention, methods for the treatment of cancer are provided comprising administering a therapeutically effective amount of an HDAC6 inhibitor, as described herein, to a subject in need thereof. In certain embodiments, the subject is identified as in need of such treatment. In certain embodiments, a method for the treatment of a diseases is provided comprising administering a therapeutically effective amount of an HDAC6 inhibitor, or a pharmaceutical composition comprising an HDAC6 inhibitor to a subject in need thereof, in such amounts and for such time as is necessary to achieve the desired result. In certain embodiments of the present invention a “therapeutically effective amount” of an HDAC6 inhibitor or pharmaceutical composition is that amount effective for modulating a subset of a group of cytokines according to the invention.

In certain embodiments, the method involves the administration of a therapeutically effective amount of an HDAC6 inhibitor or a pharmaceutically acceptable derivative thereof to a subject (including, but not limited to a human or animal) in need of it (including a subject identified as in need). In certain embodiments, the HDAC6 inhibitors are useful for the treatment of cancer (including, but not limited to, glioblastoma, retinoblastoma, breast cancer, cervical cancer, colon and rectal cancer, leukemia (e.g., CML, AML, CLL, ALL), lymphoma, lung cancer (including, but not limited to small cell lung cancer), melanoma and/or skin cancer, multiple myeloma, non-Hodgkin's lymphoma, ovarian cancer, pancreatic cancer, prostate cancer and gastric cancer, bladder cancer, uterine cancer, kidney cancer, testicular cancer, stomach cancer, brain cancer, liver cancer, or esophageal cancer, melanoma or multiple melanoma). In certain embodiments, the HDAC6 inhibitors of the invention are active against leukemia cells and melanoma cells, and thus are useful for the treatment of leukemias (e.g., myeloid, lymphocytic, myelocytic and lymphoblastic leukemias) and malignant melanomas. In still other embodiments, the inventive anticancer agents are active against solid tumors. Accordingly, in yet another aspect, according to the methods of treatment of the present invention, tumor cells are killed, or their growth is inhibited by contacting said tumor cells with an HDAC6 inhibitor, as described herein.

In certain embodiments, the invention provides a method of treatment of any of the disorders described herein, wherein the subject is a human.

In accordance with the foregoing, the present invention further provides a method for preventing or treating any of the diseases or disorders described above in a subject in need of such treatment, which method comprises administering to said subject a therapeutically effective amount of an HDAC6 inhibitor of the invention or a pharmaceutically acceptable salt thereof. For any of the above uses, the required dosage will vary depending on the mode of administration, the particular condition to be treated and the effect desired.

The invention provides for methods of treating any disease associated with an increase in the extracellular level of one or more cytokines using an HDAC inhibitor that modulates the extracellular level of at least one of the cytokines in the section entitled “cytokines”. In one embodiment, the invention provides for treating any one of rheumatoid arthritis, multiple sclerosis, inflammatory bowel disease, psoriasis and systemic lupus erythematosis. In one embodiment an HDAC inhibitor treats rheumatoid arthritis by modulating the extracellular level of at least one of the cytokines described herein in the section entitled “cytokines”, including but not limited to at least one of the cytokines presented in Table 5. In one embodiment an HDAC inhibitor treats multiple sclerosis by modulating the extracellular level of at least one of the cytokines described herein in the section entitled “cytokines”, including but not limited to at least one of the cytokines presented in Table 6.

In one embodiment an HDAC inhibitor treats inflammatory bowel disease by modulating the extracellular level of at least one of the cytokines described herein in the section entitled “cytokines”, including but not limited to at least one of the cytokines presented in Table 7. In one embodiment an HDAC inhibitor treats psoriasis by modulating the extracellular level of at least one of the cytokines described herein in the section entitled “cytokines”, including but not limited to at least one of the cytokines presented in Table 8. In one embodiment an HDAC inhibitor treats systemic lupus erythematosis by modulating the extracellular level of at least one of the cytokines described herein in the section entitled “cytokines”, including but not limited to at least one of the cytokines presented in Table 9.

VI. Dosages and Modes of Administration

In general, HDAC inhibitors, for example HDAC6 inhibitors of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight (0.05 to 4.5 mg/m²). An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

In certain embodiments, a therapeutic amount or dose of an HDAC6 inhibitor of the present invention may range from about 0.1 mg/kg to about 500 mg/kg (about 0.18 mg/m² to about 900 mg/m²), alternatively from about 1 to about 50 mg/kg (about 1.8 to about 90 mg/m²). In general, treatment regimens according to the present invention comprise administration to a patient in need of such treatment from about 1 mg to about 5000 mg of the compound(s) of this invention per day in single or multiple doses. Therapeutic amounts or doses will also vary depending on route of administration, as well as the possibility of co-usage with other agents.

Upon improvement of a subject's condition, a maintenance dose of an HDAC6 inhibitor, either alone of in combination with one or more additional therapeutic agents, may be administered, if necessary. Subsequently, the dosage or frequency of administration, or both, may be reduced, as a function of the symptoms, to a level at which the improved condition is retained and when the symptoms have been alleviated to the desired level, treatment should cease. The subject may, however, require intermittent treatment on a long-term basis upon any recurrence of disease symptoms.

It will be understood, however, that the total daily usage of an HDAC6 inhibitor of the present invention will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

It is contemplated that global administration of a therapeutic composition to a subject is not needed in order to achieve a highly localized effect. Localized administration of a therapeutic composition according to the invention is preferably by injection, catheter or by means of a drip device, drug pump or drug-saturated solid matrix from which the composition can diffuse implanted at the target site. When a tissue that is the target of treatment according to the invention is on a surface of an organism, topical administration of a pharmaceutical composition is possible. For example, antibiotics are commonly applied directly to surface wounds as an alternative to oral or intravenous administration, which methods necessitate a much higher absolute dosage in order to counter the effect of systemic dilution, resulting both in possible side-effects in otherwise unaffected tissues and in increased cost.

Systemic administration of a therapeutic composition according to the invention may be performed by methods of whole-body drug delivery well known in the art. These include, but are not limited to, intravenous drip or injection, subcutaneous, intramuscular, intraperitoneal, intracranial and spinal injection, ingestion via the oral route, inhalation, trans-epithelial diffusion (such as via a drug-impregnated, adhesive patch) or by the use of an implantable, time-release drug delivery device. Note that injection may be performed either by conventional means (i.e. using a hypodermic needle) or by hypospray (see Clarke and Woodland, 1975, Rheumatol. Rehabil., 14: 47-49).

Systemic administration is advantageous when a pharmaceutical composition must be delivered to a target tissue that is widely-dispersed, inaccessible to direct contact or, while accessible to topical or other localized application, is resident in an environment (such as the digestive tract) wherein the native activity of the nucleic acid or other agent might be compromised, e.g. by digestive enzymes or extremes of pH.

A therapeutic composition of use in the invention can be given in a single- or multiple dose. A multiple dose schedule is one in which a primary course of administration can include 1-10 separate doses, followed by other doses given at subsequent time intervals required to maintain and or reinforce the level of the therapeutic agent. Such intervals are dependent on the continued need of the recipient for the therapeutic agent, and/or the half-life of a therapeutic agent. The efficacy of administration may be assayed by monitoring the reduction in the levels of a symptom indicative or associated with atherosclerosis which it is designed to inhibit. The assays can be performed as described herein or according to methods known to one skilled in the art.

A therapeutically effective regimen may be sufficient to arrest or otherwise ameliorate symptoms of a disease. An effective dosage regimen requires providing the regulatory drug over a period of time to achieve noticeable therapeutic effects wherein symptoms are reduced to a clinically acceptable standard or ameliorated. The symptoms are specific for the disease in question. For example, when the disease is associated with tumor formation, the claimed invention is successful when tumor growth is arrested, or tumor mass is decreased by at least 50% and preferably 75%.

VI. Pharmaceutical Compositions

In another aspect, the invention provides a pharmaceutical composition comprising an HDAC6 inhibitor, or a pharmaceutically acceptable ester, salt, or prodrug thereof, together with a pharmaceutically acceptable carrier.

HDAC6 inhibitors of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising an HDAC6 inhibitor of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

HDAC6 inhibitors of the invention can be administered in therapeutically effective amounts in combination with one or more therapeutic agents (pharmaceutical combinations). For example, synergistic effects can occur with other anti-proliferative, anti-cancer, immunomodulatory or anti-inflammatory substances. Where the compounds of the invention are administered in conjunction with other therapies, dosages of the co-administered compounds will of course vary depending on the type of co-drug employed, on the specific drug employed, on the condition being treated and so forth.

Combination therapy includes the administration of an HDAC6 inhibitor in further combination with other biologically active ingredients (such as, but not limited to, a second and different antineoplastic agent) and non-drug therapies (such as, but not limited to, surgery or radiation treatment). For instance, the HDAC6 inhibitors of the invention can be used in combination with other pharmaceutically active compounds, preferably compounds that are able to enhance the effect of the compounds of the invention. The HDAC6 inhibitors of the invention can be administered simultaneously (as a single preparation or separate preparation) or sequentially to the other drug therapy. In general, a combination therapy envisions administration of two or more drugs during a single cycle or course of therapy.

In certain embodiments, these compositions optionally further comprise one or more additional therapeutic agents. Alternatively, a compound of this invention may be administered to a patient in need thereof in combination with the administration of one or more other therapeutic agents. For example, additional therapeutic agents for conjoint administration or inclusion in a pharmaceutical composition with a compound of this invention may be an approved chemotherapeutic agent, or it may be any one of a number of agents undergoing approval in the Food and Drug Administration that ultimately obtain approval for the treatment of a disease including but not limited to any of the diseases recited herein. In certain other embodiments, the additional therapeutic agent is an anticancer agent. In certain other embodiments, the compositions of the invention are useful for the treatment of protozoal infections. In the treatment of cancer or protein degradation disorders, the inventive compound may be combined with a proteasome inhibitor (e.g., bortezomib, RI 15777 FTI, MG132, NPI-0052, etc.). In the treatment of cancer or protein degradation disorders, the inventive compound may be combined with protein degradation inhibitor (e.g. another compound, for example, a tubacin-like compound, bortezomib, RI 15777 FTI, MGI 32, NPI-0052, SAHA, ¹⁶⁶Ho-DOTMP, arsenic trioxide, 17-AAG, MG 132, etc.).

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

The present invention encompasses pharmaceutically acceptable topical formulations of inventive compounds. The term “pharmaceutically acceptable topical formulation,” as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to, solvents {e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et ah, Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, IU. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide. fatty acid esters (e.g., isopropyl myristate, methyl laurate, glycerol monooleate, and propylene glycol monooleate) and N-methylpyrrolidine.

In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another immunomodulatory agent, anticancer agent or agent useful for the treatment of psoriasis), or they may achieve different effects (e.g., control of any adverse effects).

For example, other therapies or anticancer agents that may be used in combination with the inventive compounds of the present invention include, but not limited to, surgery, radiotherapy (in but a few examples, gamma-radiation, neutron beam radiotherapy, electron beam radiotherapy, proton therapy, brachytherapy, and systemic radioactive isotopes, to name a few), endocrine therapy, biologic response modifiers (interferons, interleukins, antibodies, aptamers, siRNAs, oligonucletoides, enzyme, ion channel and receptor inhibitors or activators to name a-few), hyperthermia and cryotherapy, agents to attenuate any adverse effects (e.g., antiemetics), and other approved chemotherapeutic drugs, including, but not limited to, alkylating drugs (e.g., mechlorethamine, chlorambucil, Cyclophosphamide, Melphalan, Ifosfamide), antimetabolites (e.g., Methotrexate), purine antagonists and pyrimidine antagonists (e.g., 6-Mercaptopurine, 5-Fluorouracil, Cytarabile, Gemcitabine), spindle poisons (e.g., Vinblastine, Vincristine, Vinorelbine, Paclitaxel), podophyllotoxins (e.g., Etoposide, Irinotecan, Topotecan), antibiotics (Doxorubicin, Bleomycin, Mitomycin), nitrosoureas (e.g., Carmustine, Lomustine), inorganic ions (e.g., Cisplatin, Carboplatin), enzymes (e.g., Asparaginase), and hormones (e.g., Tamoxifen, Leuprolide, Flutamide, and Megestrol), to name a few. For a more comprehensive discussion of updated cancer therapies see, The Merck Manual, Seventeenth Ed. 1999, the entire contents of which are hereby incorporated by reference. See also the National Cancer Institute (CNI) website (www.nci.nih.gov) and the Food and Drug Administration (FDA) website for a list of the FDA approved oncology drugs (www.fda.gov/cder/cancer/dmglistframe).

In certain embodiments, the pharmaceutical compositions of the present invention further comprise one or more additional therapeutically active ingredients (e.g., chemotherapeutic and/or palliative). For purposes of the invention, the term “palliative” refers to treatment that is focused on the relief of symptoms of a disease and/or side effects of a therapeutic regimen, but is not curative. For example, palliative treatment encompasses painkillers, antinausea medications, anti-pyretics, and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

The present compounds and compositions can be administered together with hormonal, steroidal anti-inflammatory agents, such as but not limited to, estradiol, conjugated estrogens (e.g., PREMARIN, PREMPRO, AND PREMPHASE), 17 beta estradiol, calcitonin-salmon, levothyroxine, dexamethasone, medroxyprogesterone, prednisone, cortisone, flunisolide, and hydrocortisone; non-steroidal anti-inflammatory agents, such as but not limited to, tramadol, fentanyl, metamizole, ketoprofen, naproxen, nabumetone, ketoralac, tromethamine, loxoprofen, ibuprofen, aspirin, and acetaminophen; disease-modifying antirheumatic agents (DMARDs), such as but not limited to, methotrexate, biologic disease-modifying anti-rheumatic agents, such as but not limited to, anti-TNF-α antibodies, such as infliximab (REMICADE™) and adalimumab (Humira™), fusion proteins containing the ligand-binding domain of TNF-α, such as etanercept (ENBREL™), and interleukin-1 (IL-1) receptor antagonist, such as anakinra (KINERET™).

The pharmaceutical compositions of the present invention comprise a therapeutically effective amount of a compound of the present invention formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. The pharmaceutical compositions of this invention can be administered to humans and other animals orally, rectally, parenterally, intracisternally, intravaginally, intraperitoneally, topically (as by powders, ointments, or drops), buccally, or as an oral or nasal spray.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present invention, disorders are treated or prevented in a subject, such as a human or other animal, by administering to the subject a therapeutically effective amount of an HDAC6 inhibitor of the invention, in such amounts and for such time as is necessary to achieve the desired result. The term “therapeutically effective amount” of a compound of the invention, as used herein, means a sufficient amount of the compound so as to decrease the symptoms of a disorder in a subject. As is well understood in the medical arts a therapeutically effective amount of a compound of this invention will be at a reasonable benefit/risk ratio applicable to any medical treatment.

The invention also provides for a pharmaceutical combinations, e.g. a kit, comprising a first agent which is a compound of the invention as disclosed herein, in free form or in pharmaceutically acceptable salt form. The kit can comprise instructions for its administration to a subject suffering from or susceptible to a disease or disorder. The kit may also comprise a second therapeutic agent.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g., a compound of the invention and a co-agent, are both administered to a patient simultaneously in the form of a single entity or dosage. The term “non-fixed combination” means that the active ingredients, e.g., a compound of the invention and a co-agent, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols; such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The protein kinase inhibitors or pharmaceutical salts thereof may be formulated into pharmaceutical compositions for administration to animals or humans. These pharmaceutical compositions, which comprise an amount of the protein inhibitor effective to treat or prevent a protein kinase-mediated condition and a pharmaceutically acceptable carrier, are another embodiment of the present invention.

This invention also encompasses pharmaceutical compositions containing, and methods of treating disorders through administering, pharmaceutically acceptable prodrugs of HDAC6 inhibitors of the invention. For example, HDAC6 inhibitors of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxyysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxy carbonyls, as outlined in Advanced Drug Delivery Reviews, 1996, 19, 1 15. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. Particularly, in embodiments the compound is at least 85% pure, more preferably at least 90% pure, more preferably at least 95% pure, and most preferably at least 99% pure.

VII. Kits or Pharmaceutical Systems

The present compositions may be assembled into kits or pharmaceutical systems for use in modulation of a subset of cytokines and disease treatment. Kits or pharmaceutical systems according to this aspect of the invention comprise a carrier means, such as a box, carton, tube or the like, having in close confinement therein one or more container means, such as vials, tubes, ampules, bottles and the like. The kits or pharmaceutical systems of the invention may also comprise associated instructions for using the agents of the invention, for example an HDAC inhibitor that modulates a subset of cytokines. The HDAC inhibitors of the kits or pharmaceutical systems of the invention may have any one of the functional properties described for the HDAC inhibitors of the methods of the invention.

VII. Uses

The methods of the invention can be used to treat a subject with a disease. The methods of the invention can also be used for monitoring the treatment of a subject with a disease. The methods of the invention are also useful for modulating the extracellular level of a group of cytokines or a subset thereof. The methods of the invention can be used to design a treatment protocol for a subject diagnosed with a disease. The invention also provides for pharmaceuticals that can be used to treat a disease. The methods of the invention are also useful for screening for an HDAC6 inhibitor.

VIII. Animal Models

The invention provides for animal models for various diseases including but not limited to rheumatoid arthritis, multiple sclerosis, inflammatory bowel disease, and Type I diabetes. Additional animal models known in the art are also useful according to the invention.

A. Rheumatoid Arthritis:

Animal models for Rheumatoid arthritis include but are not limited to collagen induced arthritis in mouse and rat, collagen antibody induced arthritis in mouse, spontaneous rheumatoid arthritis in K/BxN mice, arthritis induced by adoptive transfer of serum from K/BxN mice and spontaneous arthritis in TNFα transgenic mice.

B. Multiple Sclerosis:

Animal models for Multiple Sclerosis include but are not limited to experimental autoimmune encephalopathy in mouse and rat induced by injection of myelin oligodendrocyte glycoprotein and experimental autoimmune encephalopathy in mouse and rat induced by injection of proteolipid protein.

C Inflammatory Bowel Disease (Crohn's Disease):

Animal models for Crohn's Disease include but are not limited to Dextran sodium sulfate induced colitis in mouse and rat and colitis induced by adoptive transfer of CD4+CD45RBhigh cells into SCID mice

D. Inflammatory Bowel Disease (Ulcerative Colitis):

An animal model for ulcerative colitis includes but is not limited to trinitrobenzene sulfonic acid induced colitis in mouse and rat.

E. Type I Diabetes: Spontaneous Type I Diabetes

An animal model for Type I Diabetes includes but is not limited to BB/Wor rat or NOD mice.

F. Graft Versus Host Disease

An animal model for graft versus host disease includes but is not limited to transfer of allogenic donor lymphocytes and stem cells into irradiated host mice and transfer of allogenic donor lymphocytes and stem cells into immune competent host mice.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

Having now generally described the invention, the same will be more readily understood through reference to the following Examples which are provided by way of illustration, and are not intended to be limiting of the present invention, unless specified.

The following examples are put forth for illustrative purposes only and are not intended to limit the scope of what the inventors regard as their invention.

### Example 1

### HDAC Inhibitor Modulation of LPS Induced Cytokine Release from PBMCs

The effect of HDAC inhibitors on LPS induced cytokine release from PBMCs was determined as follows.

### PBMC Isolation and Stimulation

Whole blood from three anonymous donors is collected in vacutainer tubes with sodium heparin as an anti-coagulant (Research Blood Components, Brighton, Mass.). A total volume of 30 ml of blood is diluted and layered over Ficoll-Paque Plus (GE Life Sciences) and centrifuged at 400×g for 30 minutes. The peripheral blood mononuclear cell (PBMC) fraction is removed from the interface of plasma and Ficoll and washed once with PBS. The cells are resuspended in RPMI-1640 supplemented with 10% fetal bovine serum.

Cells are seeded in 24 well plates at a density of 400,000 cells per well. The cells are treated with 1 μM of the HDAC inhibitors SAHA, ACY-216, ACY-257, or dexamethasone (Sigma, catalog number D2915), or DMSO, for one hour. After this incubation the cells are stimulated with 0.5 ng/ml lipopolysaccharide (LPS, Sigma catalog number L-8274).

After the overnight stimulation the plates are centrifuged at 200×g for 5 minutes. The supernatant is removed and a small aliquot is reserved for determination of TNFα levels by ELISA (R&D Systems, catalog number STA00C). The remainder of the supernatant is frozen at −80° C. and sent to Millipore Bioscience Division (St. Charles, Mo.) for multiplex cytokine analysis.

### Cytokine Profiling

Millipore tests the supernatants for 14 cytokines using Luminex beads. Table 10 shows the level of cytokines in the supernatant of unstimulated PBMC and PBMC stimulated with 0.5 ng/ml LPS.

TABLE 10
Level of 14 cytokines in supernatant of human PBMCs
stimulated overnight with 0.5 ng/ml LPS. Values are
in pg/ml. BLQ = below limit of quantitation.

| Cytokine | Unstimulated | LPS |
|---|---|---|
| GM-CSF | BLQ | 15.4 |
| IFNγ | BLQ | BLQ |
| IL-1β | BLQ | 222 |
| IL-2 | BLQ | BLQ |
| IL-4 | BLQ | BLQ |
| IL-5 | BLQ | BLQ |
| IL-6 | 32.9 | 2891 |
| IL-7 | 5.6 | 26.9 |
| IL-8 | 1902 | 3729 |
| IL-10 | 6.9 | 284 |
| IL-12 | BLQ | BLQ |
| IL-13 | BLQ | BLQ |
| MCP-1 | 1576 | 2750 |
| TNFα | 11.4 | 365 |

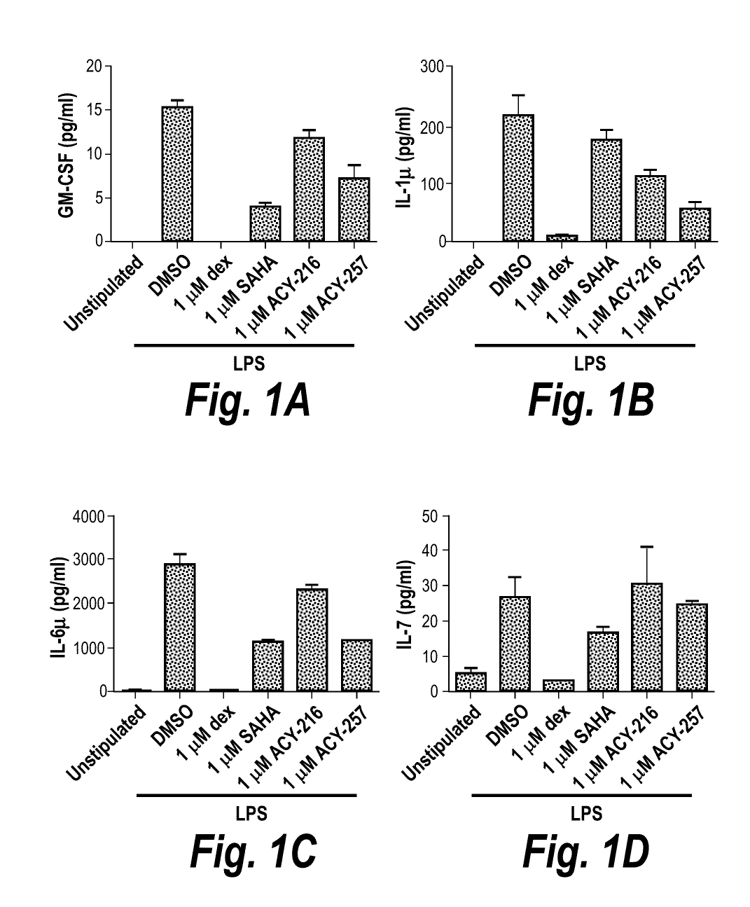

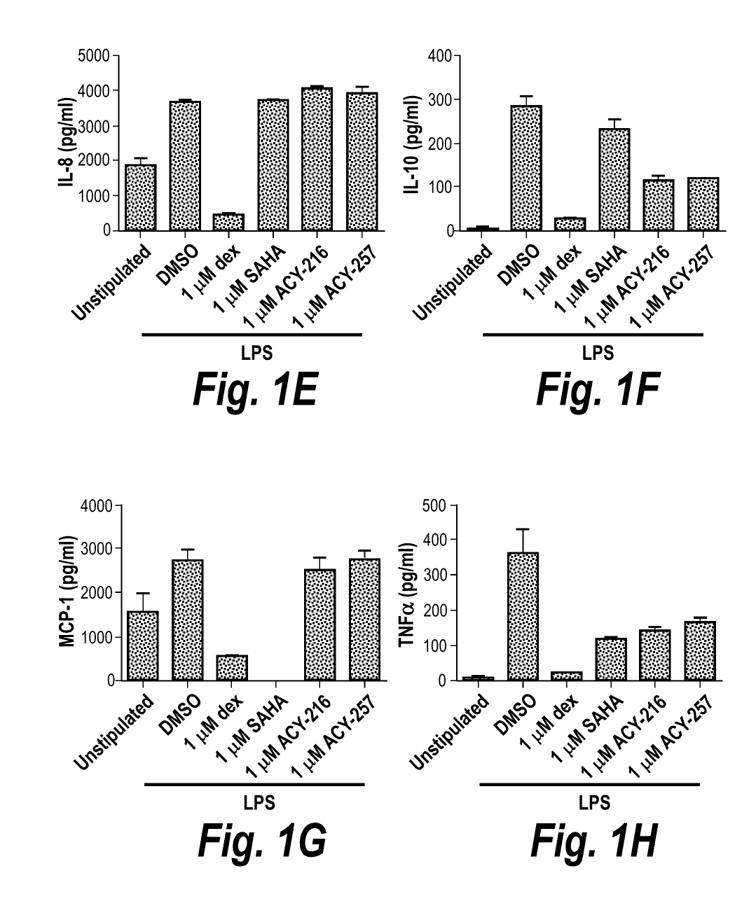

The results of that analysis are shown in FIG. 1 which presents the effects of HDAC inhibitory compounds on the level of the cytokines in the supernatants. While dexamethasone decreases the production of all cytokines induced by LPS, SAHA decreases six out of eight cytokines, ACY-216 effects three of eight, and ACY-257 effects five of eight. The highly selective HDAC6 inhibitor ACY-257 has no effect on the production of IL-7, IL-8 and MCP-1, while significantly decreasing GM-CSF, IL-1β, IL-6, IL-10, and TNF-α.

HDAC inhibitory activity of the compounds described in Table 1 is measured using a kinetic HDAC assay. Recombinant HDACs are obtained from BPS Biosciencies (San Diego, Calif.). Fluorophore conjugated substrates are synthesized at Chempartner (Shanghai, China). Reactions are performed in assay buffer (50 mM HEPES, 100 mM KCl, 0.001% Tween-20, 0.05% BSA, 200 μM TCEP, pH 7.4) and followed for fluorogenic release of 7-amino-4-methylcoumarin from substrate upon dacetylase and trypsin enzymatic activity. Fluorescence measurements are obtained approximately once per minute using a multilabel plate reader. Data were analyzed using GraphPad Prism.

### Example 2

### HDAC Inhibitor Modulation of Cytokine Release from Macrophages

The effect of HDAC inhibitors on cytokine release from macrophages is determined as follows.

### Monocyte Derived Macrophage Isolation and Stimulation

Monocytes are separated from whole human blood using RosetteSep negative selection (Stem Cell Technologies). Monocytes are incubated in StemSpan H300 media supplemented with 20 ng/ml CSF-1 in 24 well plates. After one week the cells will take on the appearance of macrophages. The cells are pre-incubated with various concentrations of Trichostatin A, ACY-63, ACY-251 and ACY-257. After a one hour pre-incubation the cells are stimulated with 10 ng/ml LPS overnight. The supernatant is removed and assayed for TNF-α content by ELISA and the supernatants sent to Millipore for multiplex cytokine analysis.

Inhibition of Cytokine Release from Macrophages

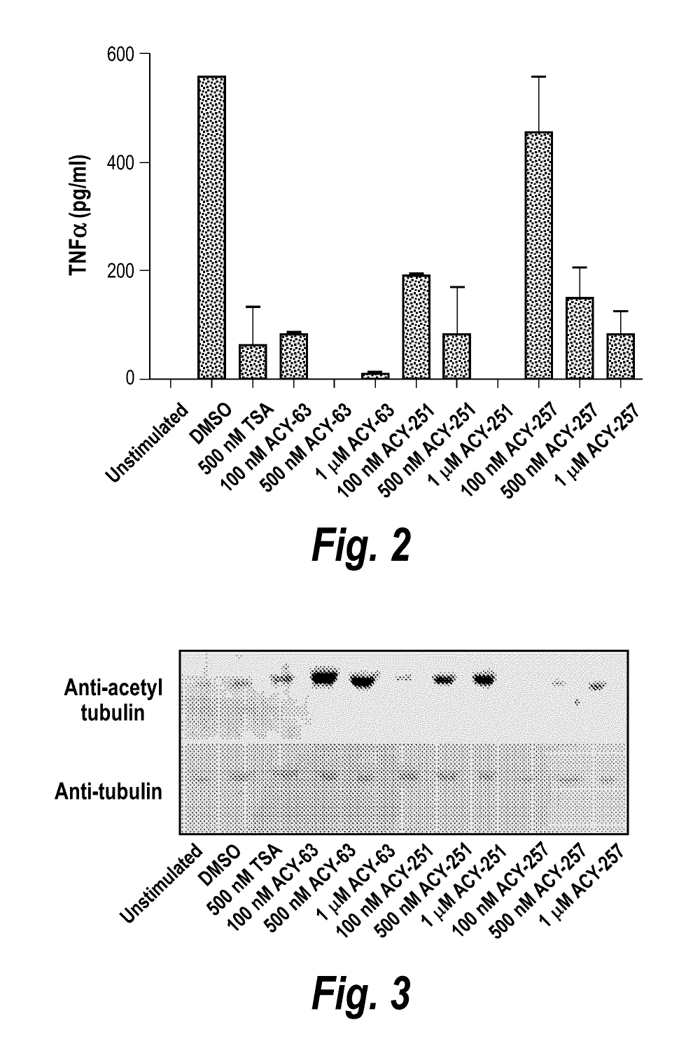

To determine if HDAC6 inhibition can prevent cytokine release from macrophages, human monocytes are differentiated into macrophages in vitro by treating them for one week with CSF-1. The cells are treated with four HDAC inhibitors, Trichostatin A (non selective), ACY-63 (ten fold selective for HDAC6 versus HDAC3), ACY-251 (30 fold selective), or ACY-257 (70 fold selective). The cells are stimulated overnight with 10 ng/ml LPS. The supernatant is collected and the level of TNF-α is determined by ELISA. The results are shown in FIG. 2.

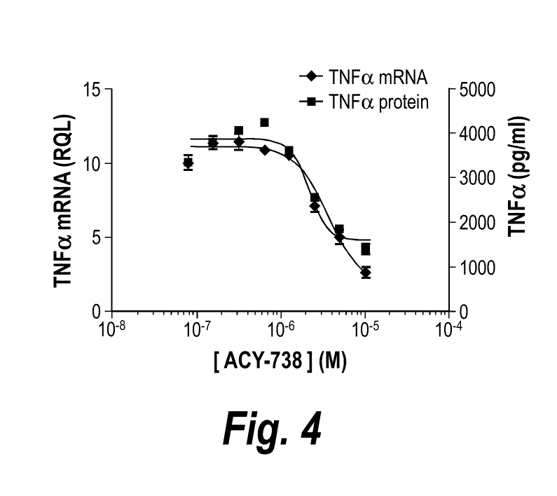

To determine if inhibition of cytokine release is a result of a decrease in mRNA levels, RAW264.7 cells are treated with various concentrations of an HDAC inhibitor ACY-738 for two hours. The cells are stimulated with 10 ng/ml LPS and incubated for two hours. The cell supernatants are collected and assayed for the level of TNFα by ELISA. The cells are harvested and mRNA purified using an RNEasy kit from Qiagen. The level of TNFα mRNA is determined by RT-PCR using a StepOne Plus real time PCR machine. The change in the relative level of TNFα mRNA as compared to the level of GAPDH mRNA is plotted, along with the concentration of TNFα protein in the supernatant (see FIG. 4).

The HDAC inhibitor ACY-739 decreases both the protein and mRNA level of TNFα.

### Western Blotting

Cell lysates prepared in SDS-PAGE sample buffer are run on a 4-20% gel and transferred to nitrocellulose. The blots are probed with anti-acetyl tubulin (monoclonal antibody 6-11b-1) and anti-mouse alkaline phosphatase. The intensity of the bands is measured using a Syngene G:box system.

To correlate the inhibition of TNF-α release with HDAC6 inhibition, the cells are harvested in SDS-PAGE sample buffer and the level of acetyl-tubulin is measured by Western Blot as shown in FIG. 3.

The inhibition of TNF-α release correlates well with the acetylation of tubulin, which is a marker of HDAC6 inhibition.

### Example 3

### HDAC Inhibitor Modulation of TNF-α Production In Vivo

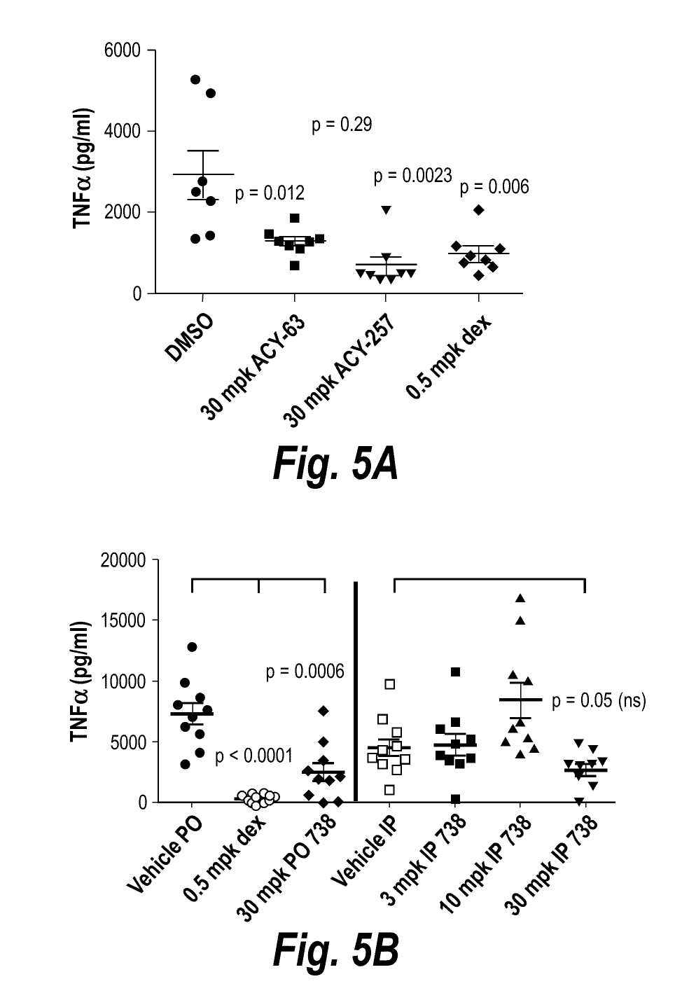

To determine if the ability of ACY-63 and ACY-257 to inhibit TNF-α production occurs in vivo, Swiss Webster mice are treated with either ACY-63 or ACY-257 at 30 mg/kg by IP injection. Four hours post dose the mice are challenged with 0.5 mg/kg of LPS. Ninety minutes after the LPS challenge the mice are anesthetized and bled by cardiac puncture. The blood is collected in EDTA and plasma is separated. The plasma is analyzed for the concentration of TNF-α by ELISA. The results are shown in FIG. 5a.

Inhibition of TNF-in the plasma by inhibition by HDAC6 selective compounds was determined in mice challenged with LPS. Compounds were administered by either oral gavage or IP injection two hours prior to LPS challenge, n=10. p values calculated with unpaired two-tailed t-test. Individual data points with mean+/−SEM (FIG. 5b).

To determine if an HDAC inhibitor can inhibit TNF-α production in vivo when given by an oral route, Swiss Webster mice are treated with ACY-738 either by oral gavage or IP injection. Two hours post dose the mice are challenged with 0.5 mg/kg of LPS. Ninety minutes after the LPS challenge the mice are anesthetized and bled by cardiac puncture. The blood is collected in EDTA and plasma is separated. The plasma is analyzed for the concentration of TNF-α by ELISA. The results are shown in FIG. 4.

The results in FIGS. 4 and 5 demonstrate that selective HDAC6 inhibition can reduce cytokine levels in vivo.

### Example 4

### The Effect of HDAC6 Inhibitors on Chronic Inflammatory Disease

The effect of HDAC6 inhibitors on chronic inflammatory disease was tested as follows.

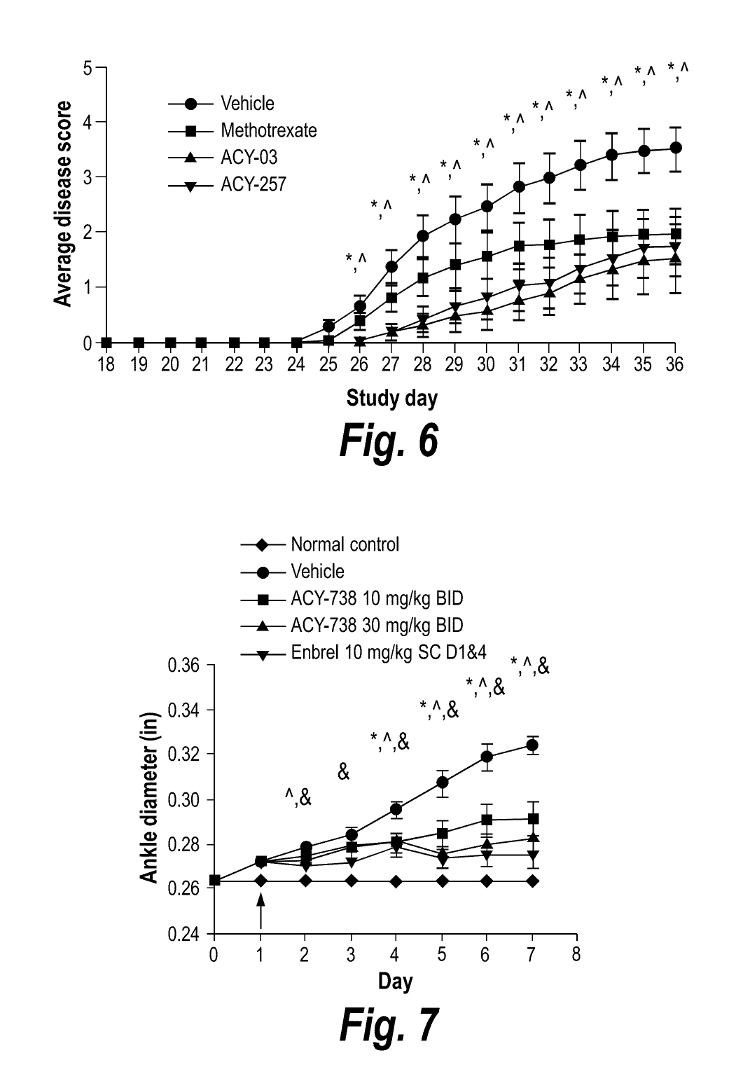

To test these two HDAC6 inhibitors in an animal model of a chronic inflammatory disease a mouse collagen induced arthritis in a semi-therapeutic mode is used. In this study male DBA/1OlaHsd mice, 6 weeks old, are anesthetized with Isoflurane and given 150 μL Type II collagen in Freund's complete adjuvant on Day 0 and Day 21. Treatment with compound is initiated on Day 18. The mice in the treatment groups are injected twice daily with 30 mg/kg of compound in 10% DMSO/10% Solutol HS15/80% 0.9% saline. At day 28 the injections are decreased to one per day due to weight loss in all groups. Inflammation in the joints is measured daily and a disease score is calculated based on paw thickness. The results of this experiment are shown in FIG. 6. FIG. 6 presents the effect of HDAC inhibitors on the development of collagen induced arthritis in mice. n=12 (control and methotrexate), n=11 (ACY-257), n=10 (ACY-63). Mean+/−SEM. * ACY-63 versus vehicle p<0.05. ̂ ACY-257 versus vehicle p<0.05 (unpaired two tailed t-test).

FIG. 6 demonstrates that the selective HDAC6 inhibitors decrease the disease score in collagen induced arthritis in mice. The compounds are more efficacious than methotrexate in this experiment. In this model the mice are dosed after the initial induction of disease, but before the boost on day 21 and before the first onset of disease. The compounds delay the appearance of disease by two days and decrease the severity of disease by an average of 67% for ACY-63 and 50% for ACY-257.

To further test if HDAC6 inhibitors decrease disease in a model of chronic inflammation, ACY-738 is tested in rats with collagen induced arthritis. In this study female Lewis rats are anesthetized with Isoflurane and given 300 μL Type II collagen in Freund's complete adjuvant on Day 0 and Day 6. Treatment with compound is initiated when ankle swelling becomes measurable, nominally day 10. The rats in the treatment groups are given either 10 mg/kg or 30 mg/kg ACY-738 in 5% DMSO/95% D5W twice daily by oral gavage. One group of rats is treated with Enbrel by subcutaneous injection on the first and fourth day of measurable ankle swelling. Inflammation in the joints and body weight are measured. The results of this experiment are shown in FIG. 7.

FIG. 7 presents the effect of HDAC6 inhibitors on the development of collagen induced arthritis in rats. n=Effect of HDAC6 inhibitors on the development of collagen induced arthritis in rats. n=4 (normal control), n=8 (ACY-738 and Enbrel). Mean+/−SEM. Compound treatment started at Day 1 when all induced animals showed ankle swelling. * ACY-738 10 mg/kg versus vehicle p<0.05, ̂ ACY-738 30 mg/kg versus vehicle p<0.05, & Enbrel versus vehicle p<0.05 (unpaired two tailed t-test).

1. A method of treating a subject with a disease, comprising:
identifying a subject in need of treatment;
administering to said subject an HDAC6 inhibitor;
determining the extracellular level of a group of cytokines;
wherein following said administration, there is a modulation of the extracellular level of a subset of cytokines of said group, thereby treating said disease.

2-3. (canceled)

4. A method of treating a subject with a disease according to claim 1, additionally comprising monitoring the treatment of a subject diagnosed with a disease, by:
comparing the extracellular level of said group of cytokines of said subject before and after administration of said HDAC6 inhibitor wherein following administration of said HDAC6 inhibitor there is a modulation of the extracellular level of a subset of said group of cytokines thereby indicating treatment.

5-8. (canceled)

9. The method of claim 1, wherein modulation comprises a decrease in the extracellular level of at least one cytokine of said group.

10. The method of claim 1, wherein modulation comprises a decrease in the extracellular level of at least one cytokine of said group and an increase in the extracellular level of at least one cytokine of said group.

11. The method of claim 9 wherein modulation comprises a decrease in the extracellular level of at least one cytokine of said group, and wherein an increased extracellular level of said at least one cytokine is associated with a disease.

12. The method of claim 9, wherein the extracellular level of at least one cytokine of said group is not modulated.

13-17. (canceled)

18. A method of modulating the extracellular level of a subset of a group of cytokines,
comprising contacting a cell with an inhibitor of HDAC6; and
determining the extracellular level of said group of cytokines,
wherein the extracellular level of a subset of said group of cytokines is modulated following said contacting.

19. (canceled)

20. The method of claim 1, wherein at least one side effect selected from the group consisting of nausea, vomiting, diarrhea, fatigue, QT/QTc prolongation, torsades de point, cardiac arrhythmias, myelosuppression, depressed blood cell count, and thrombocytopenia, lymphopenia, neutropenia is not detected.

21. The method of claim 1, wherein a second therapeutic agent is administered to said subject with said HDAC6 inhibitor.

22-29. (canceled)

30. The method of claim 1 or 18, wherein said group of cytokines comprises at least one of said cytokines of any one of Tables, 3, 5, 6, 7, 8 and 9 and wherein said group of cytokines also comprises at least one of said cytokines of Table 4.

31. The method of claim 1 or 18, wherein said group of cytokines comprises at least one of said cytokines in any one of Tables 5, 6, 7, 8, and 9.

32. The method of claim 1 or 18, wherein said group of cytokines comprises TNF-α.

33. The method of claim 1, wherein said disease is selected from the group consisting of: rheumatoid arthritis, multiple sclerosis, inflammatory bowel disease, psoriasis, and systemic lupus erythematosis.

34. The method of claim 1 or 18, wherein said HDAC6 inhibitor is specific for HDAC6.

35. The method of claim 1, wherein said HDAC6 inhibitor is administered in a therapeutically effective amount or a pharmaceutically acceptable salt or prodrug thereof, or a pharmaceutical composition comprising a therapeutically effective amount or a pharmaceutically acceptable salt or prodrug thereof, to the subject, thereby treating said disease.

36. (canceled)

37. The method of claim 1, wherein said subject is a mammal.

38. The method of claim 1, wherein said subject is a human.

39. (canceled)

40. The method of claim 35, wherein said therapeutically effective amount of said HDAC6 inhibitor is administered by topical application, intravenous drip or injection, subcutaneous, intramuscular, intraperitoneal, intracranial and spinal injection, ingestion via oral route, inhalation, trans-epithelial diffusion or an implantable, time-release drug delivery device.

41-42. (canceled)

43. A packaged pharmaceutical comprising:
(a) an HDAC inhibitor or a pharmaceutically acceptable salt or prodrug thereof and
(b) associated instructions for using said HDAC inhibitor to treat a disease associated with an increase in the extracellular level of a group of cytokines.

44-46. (canceled)

47. A method of screening for an inhibitor of HDAC6 comprising the steps of:
determining the extracellular levels of a group of cytokines of a cell;
contacting said cell with a compound;
comparing the extracellular level of said group of cytokines before and after said contacting step; and wherein a compound that modulates the extracellular level of a subset of said group of cytokines is identified as an inhibitor of HDAC6 inhibitor.

48. (canceled)
